(19) United States
(12) Patent Application Publication
(10) Pub. No.: US 2008/0274991 A1
(43) Pub. Date: Nov. 6, 2008

(54) Nucleic Acids Against Viruses, in Particular Hiv

(76) Inventors: Benjamin Berkhout, Naarden (NL); Olivier Ter Brake, Amsterdam (NL)

Correspondence Address: TRASK BRITT, P.O. BOX 2550, SALT LAKE CITY, UT, 84110, US

(21) Appl. No.: 11/665,510

(22) PCT Filed: Oct. 14, 2005
(86) PCT No.: PCT/NL05/00739

§ 371 Date: Jun. 20, 2007

(30) Foreign Application Priority Data
Oct. 15, 2004 (EP) 04077855.7

(51) Int. Cl. A61K 31/70; C07H 21/04; C12N 5/06; A61P 37/00

(52) U.S. Cl. 514/44; 536/23.72; 536/24.5; 435/375

(57) ABSTRACT

The invention provides a nucleic acid molecule comprising a sequence that is complementary to a mutant of a genomic nucleic acid sequence of an organism capable of mutating its genome. The genomic nucleic acid sequence preferably comprises a conserved, essential region. Furthermore, the genomic nucleic acid sequence preferably comprises a coding sequence and a DNA/RNA regulatory sequence. A pharmaceutical composition and a gene delivery vehicle comprising a nucleic acid molecule of the invention is also provided, as well as a method for counteracting an organism comprising providing the organism with the nucleic acid molecule. The organism is preferably additionally provided with a nucleic acid sequence complementary to a genomic nucleic acid sequence of the organism.

### CROSS-REFERENCE TO RELATED APPLICATIONS

This is a national phase entry under 35 U.S.C. § 371 of International Patent Application PCT/NL2005/000739, filed Oct. 14, 2005, published in English as International Patent Publication WO 2006/041290 A2 on Apr. 20, 2006, which claims the benefit of European Patent Application Serial No. 04077855.7 filed Oct. 15, 2004, the entire contents of each of which are hereby incorporated herein by this reference.

### STATEMENT ACCORDING TO 37 C.F.R. § 1.52(e)(5)—SEQUENCE LISTING SUBMITTED ON COMPACT DISC

Pursuant to 37 C.F.R. § 1.52(e)(1)(iii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “P70419US00.5T25.txt” which is 29 KB and created on Oct. 23, 2007.

### TECHNICAL FIELD

The invention relates to biology. More specifically, the invention relates to counteracting an organism.

### BACKGROUND

Infectious diseases caused by (micro)organisms such as, for instance, bacteria and/or viruses are a common phenomenon worldwide. A problem involved with counteracting (pathogenic) organisms is their capability of developing resistance against a certain treatment. Evolvement of resistant organisms often involves alterations in the genome of such organisms. Escape mutants evolve during prolonged periods of treatment. For instance, bacteria become resistant to antibiotic treatment and escape mutant viruses evolve during antiviral therapy. In view of this, therapy directed against pathogenic organisms capable of mutating their genomes often involves a combination of approaches. In spite of such combination therapy, escape mutants are still a major concern during treatment of a variety of infectious diseases.

### DISCLOSURE OF THE INVENTION

It is an object of the present invention to provide additional means and methods for counteracting an organism capable of mutating its genome.

The invention provides a nucleic acid molecule comprising a sequence that is complementary to a mutant of a genomic nucleic acid sequence of an organism capable of mutating its genome. The invention furthermore provides a nucleic acid molecule or a binding molecule capable of specifically binding a mutant of a genomic nucleic acid sequence of an organism capable of mutating its genome, or an expression product thereof. The invention provides a different approach for avoiding and/or counteracting the occurrence of escape mutants during a certain treatment of (micro)organisms. In the art, attempts to avoid escape mutants involve a combination of several drugs/treatments that are directed towards different parts and/or different functions of a certain (micro)organism. For instance, different genomic sequences are targeted. The present invention provides the insight that a treatment is improved if a nucleic acid or any kind of binding molecule is administered that is capable of specifically binding (a gene product of) an escape mutant. Hence, escape mutants evolving during current therapies and resulting in loss of protection, are now counteracted by a nucleic acid molecule and/or binding molecule of the present invention. Therefore, protection is now maintained.

An organism is capable of mutating its genome if at least one nucleic acid sequence of its genome alters over time. This alteration can already be present at the time of the infection, or one day after infection, or after some weeks/months or it can even take years (for example, ten years) after infection before such alterations are present. The frequency and the time point at which the mutations/alteration occur depend on, for example, the virus type and/or the type of medicine(s) and/or the way in which the medicines are used. Preferably, at least one nucleic acid sequence alters during treatment as a result of selective pressure. Escape mutants are favored during a treatment targeting a wild-type organism because such treatment does not affect escape mutants. If a mutant evolves that is capable of performing all, if not most, essential functions of the organism, such mutant will be capable of outgrowing, while a wild-type organism is suppressed by the treatment. A nucleic acid molecule or binding molecule of the present invention is preferably directed towards such escape mutant. Use of the nucleic acid molecule during treatment allows for avoiding and/or counteracting the appearance of the escape mutant.

Preferably, the invention provides a therapy based on at least one nucleic acid molecule or binding molecule that comprises a sequence that is complementary to a mutant of a genomic nucleic acid sequence of an organism capable of mutating its genome, which therapy (compared to other therapies) prolongs the time period before an escape mutant emerges. Moreover, the faster an organism is capable of mutating its genome, the more important it is to avoid and/or counteract the occurrence of escape mutants with a nucleic acid molecule or binding molecule of the invention.

The organism preferably comprises a pathogenic organism. In one embodiment, the organism comprises a microorganism. Preferably, the organism comprises a virus or a bacterium. In one embodiment, the organism comprises a DNA or RNA virus, since viruses are generally well capable of developing escape mutants. More preferably, the organism comprises an RNA virus, most preferably a retrovirus. Antiviral treatment often involves occurrence of escape mutants caused by the error-prone nature of the reverse transcriptase enzyme. In one preferred embodiment, the organism comprises HIV, Hepatitis B, Hepatitis C, SARS, Ebola, Influenza, West Nile and/or polio virus. Escape mutants of these pathogens appear, for example, in case of prolonged culturing with an antiviral agent and upon prolonged therapy. A nucleic acid molecule or binding molecule of the invention is, however, suitable for inhibition of many other kinds of organisms capable of mutating their genome. By “inhibition of an organism” is meant herein that at least one function of the organism is significantly impaired. Preferably, at least one essential function of the organism is significantly impaired. In a most preferred embodiment, the organism has lost its capability of replicating and/or spreading.

A nucleic acid molecule comprises a sequence that is complementary to a mutant of a genomic nucleic acid sequence if the nucleic acid molecule comprises an anti-sense strand complementary to the sense strand of a mutant of the genomic nucleic acid sequence. The mutant comprises at least one mutation as compared to the wild-type genomic sequence. For instance, the mutant comprises at least one nucleotide alteration, substitution, deletion or addition. The mutant has preferably retained all essential functions of the organism. A nucleic acid molecule of the invention preferably comprises DNA and/or RNA. More preferably, a nucleic acid molecule of the invention comprises double-stranded RNA in order to use RNA interference to degrade RNA of an undesired organism, as explained below. In other embodiments, a nucleic acid molecule of the invention comprises other kinds of nucleic acid structures such as, for instance, a DNA/RNA helix, peptide nucleic acid (PNA), locked nucleic acid (LNA) and/or a ribozyme.

The genomic nucleic acid sequence, for instance, comprises an RNA sequence, such as an HIV genomic sequence. In that case, a mutant of the genomic RNA sequence comprises the RNA sequence with at least one mutation. A nucleic acid molecule of the present invention comprises a strand that is complementary to the mutant RNA sequence (an anti-sense strand of the mutant RNA sequence).

In case a mutant of a genomic nucleic acid sequence comprises a mutated DNA sequence, a nucleic acid molecule of the invention preferably comprises a nucleic acid sequence that is complementary to the sense strand of the mutated DNA sequence. A nucleic acid molecule that is complementary to the sense strand of a mutated DNA sequence is capable of targeting the sense strand, as well as targeting an mRNA derived from the mutated DNA sequence.

A nucleic acid molecule of the invention preferably comprises double-stranded nucleic acid, more preferably double-stranded RNA, comprising both the sense strand and the anti-sense strand of a mutant nucleic acid sequence.

A nucleic acid molecule and/or a binding molecule of the present invention is particularly suitable for counteracting an escape mutant of an undesired organism. For instance, a (risk of) disease involving the presence of a pathogenic microorganism is counteracted by administration of a nucleic acid molecule and/or a binding molecule of the present invention. Preferably, treatment of a (pathogenic) organism involves counteracting both a wild-type microorganism and at least one escape mutant thereof. An escape mutant is defined as a mutant of an organism that is capable of proliferating during a treatment that counteracts other strains of the organism. A therapy counteracting a wild-type microorganism is preferably combined with administration of a nucleic acid molecule or a binding molecule of the present invention. This way, a wild-type microorganism and at least one escape mutant thereof are counteracted, allowing long-term protection. In a preferred embodiment, a therapy targeting a certain wild-type nucleic acid sequence of a microorganism is combined with administration of a nucleic acid molecule or binding molecule of the present invention capable of specifically binding a mutant of the wild-type nucleic acid sequence. In this embodiment, the same nucleic acid sequence of a (micro)organism, or gene product thereof, remains targeted by a combined treatment of the present invention, even when alterations in the sequence occur. In a more preferred embodiment, a treatment targeting at least two different wild-type nucleic acid sequences of an organism is combined with administration of nucleic acid molecules or binding molecules of the invention capable of specifically binding a mutant of at least two different wild-type nucleic acid sequences.

The sequence of a mutant of a genomic nucleic acid sequence of an organism is determined in various ways. Various kinds of mutations are predictable. A mutant organism, for instance, needs to retain its capability of performing essential functions. Therefore, mutations in an essential region are preferably avoided. If a treatment is specifically directed to such essential region, a so-called “silent mutation” will preferably arise in order to circumvent the treatment. A “silent mutation” is a mutation that does not, or to a low extent, interfere with any (multiple) function of a nucleic acid sequence. For instance, a coding region is preferably mutated, such that the resulting codons still encode the same amino acid sequence. This is performed by a substitution in the first, second or third nucleotide of a codon, such that the resulting codon encodes the same amino acid residue. Alternatively, a nucleotide substitution resulting in a conservative amino acid substitution is allowable. A conservative amino acid substitution is defined as a substitution of one amino acid with another with generally similar properties (size, hydrophobicity, etc.), such that the overall functioning is not seriously affected. Mutations, such as nucleotide substitutions, resulting in conservative amino substitutions are readily calculated by a skilled person.

An escape mutant is also predicted by computing overall structure of nucleic acid variants. For instance, a mutation outside a certain essential region sometimes results in a significantly altered structure of the resulting mutant nucleic acid sequence. Such structure alterations are computed, for instance, using a modeling program known in the art, and a nucleic acid molecule and/or binding molecule of the invention is generated targeting a part of the resulting nucleic acid sequence that has become available and is capable of being exposed to drugs, preferably as a result of the structure alteration.

Alternatively, the sequence of a mutant of a genomic nucleic acid sequence of an organism is determined by culturing the organism during a prolonged period in the presence of a certain (candidate) drug and determining what kind of mutations arise. Subsequently, a nucleic acid molecule or a binding molecule of the invention capable of specifically binding at least one of such mutant nucleic acids, or gene product thereof, is generated. Preferably, a nucleic acid molecule of the invention is generated that comprises a sequence that is complementary to at least one of the mutants. More preferably, nucleic acid molecules of the invention are generated comprising sequences complementary to most, or all, of the mutants. In order to counteract the organism in vivo, the nucleic acid molecule(s) and/or binding molecule(s) of the invention is/are preferably administered together with the (candidate) drug.

If a treatment involves counteracting a function of a certain genomic nucleic acid sequence, at least one kind of escape mutant capable of evolving is predicted and/or determined experimentally. Subsequently, a nucleic acid molecule or a binding molecule of the invention capable of specifically binding at least one of such mutants is generated. Preferably, nucleic acid molecules and/or binding molecules of the invention are generated capable of specifically binding most, if not all, predicted and/or determined mutant nucleic acid sequences, or gene products thereof. Preferably, a nucleic acid molecule of the invention is generated that comprises a sequence that is complementary to at least one of the mutants. In one embodiment, the nucleic acid molecule and/or binding molecule is administered to a patient suffering from, or at risk of suffering from, infection by the organism. The nucleic acid molecule and/or binding molecule is preferably administered together with a treatment against a wild-type form of the organism. In that case, development of escape mutants is counteracted and/or avoided because such escape mutants are immediately targeted upon appearance. Alternatively, the nucleic acid molecule and/or binding molecule is administered once the escape mutant has evolved.

In order to efficiently counteract an organism, an essential region of the organism's genome, or a gene product thereof, is preferably targeted. If a non-essential region is targeted, escape mutants readily arise because mutations in a non-essential region mostly do not inhibit proliferation. An organism is often capable of deleting a large part of a non-essential region in order to escape from treatment, without impairing proliferation. Hence, an essential region is preferably targeted in order to significantly limit the organism's possibilities of forming escape mutants. Yet, escape mutants are reported to evolve, especially when highly mutating organisms such as RNA viruses are targeted. Therefore, a nucleic acid molecule or binding molecule of the present invention is preferably capable of specifically binding a mutant of an essential nucleic acid region of an organism, or an expression product thereof. More preferably, a nucleic acid molecule of the invention is complementary to a mutant of an essential nucleic acid region of an organism. Therapy targeting at least one essential region of a wild-type organism, together with a nucleic acid molecule and/or binding molecule of the invention targeting a mutant of at least one essential region, is capable of long-time suppression of an organism.

In order to be capable of counteracting various strains of a certain organism, a nucleic acid molecule or binding molecule of the invention is preferably capable of specifically binding at least part of a conserved region. A “conserved region” is defined as a region that is at least 90% identical in at least 50% of the (known) genomes of various strains of the same kind of organism. The conserved region is preferably at least 95%, more preferably at least 98%, most preferably 100% identical in at least 50% of the known genomes of various strains of the same kind of organism. The conserved region is preferably at least 95%, more preferably at least 98%, most preferably 100% identical in at least 75% of the known genomes of various strains of the same kind of organism. In one aspect, a nucleic acid molecule of the invention is provided that is complementary to a mutant of a genomic nucleic acid sequence of an organism, wherein the genomic nucleic acid sequence comprises at least part of a conserved region. The part of a conserved region preferably comprises at least five, more preferably at least ten, even more preferably at least 15, more preferably at least 17, even more preferably at least 18, more preferably at least 19, even more preferably at least 20, more preferably at least 22 nucleotides, and most preferably at least 23 nucleotides of the conserved region. The higher the amount of nucleotides of the part, the higher the chance that a nucleic acid molecule of the invention specifically binds a mutant of any given strain of the organism.

In the art, various methods are known for counteracting an organism by targeting a genomic nucleic acid sequence or a gene product thereof. For instance, antisense DNA oligonucleotides are used since the 1980s in order to silence gene expression. Moreover, ribozymes capable of catalyzing cleavage of target RNAs are used. A preferred method for gene silencing is RNA interference (RNAi). RNA interference is a sequence-specific RNA degradation process in the cytoplasm of eukaryotic cells that is induced by double-stranded RNA. This RNA-silencing mechanism, which was first described in Caenorhabditis elegans and Drosophila melanogaster, has many similarities with post-transcriptional gene silencing in plants. RNAi and related RNA-silencing mechanisms are believed to act as a natural defense against incoming viruses and the expression of transposable elements. Besides the antiviral function of RNAi, there is evidence that RNAi plays an important role in regulating cellular gene expression. These features have characterized RNAi both as an ancient and fundamentally important mechanism in eukaryotic cell biology.

It has been shown that the process is initiated via so-called small interfering RNAs (siRNAs) of approximately 21-23 base pairs (bp), which are cleaved from double-stranded precursor RNAs by the RnaseIII-like enzyme DICER. These siRNAs associate with various proteins to form the RNA-induced silencing complex (RISC), harboring nuclease and helicase activity. The antisense strand of the siRNA guides the RISC to the complementary target RNA, and the nuclease component cleaves the target RNA in a sequence-specific manner. Hence, double-stranded RNA is capable of inducing degradation of the homologous single-stranded RNA in a host cell.

Synthetic siRNAs of 21 bp are shown to efficiently induce RNAi-mediated gene silencing when transfected into mammalian cells. In addition, RNAi are induced in mammalian cells by intracellularly expressed short hairpin RNAs (shRNAs), preferably with a length of 19 bp, with a small loop.

Viruses are both inducers and targets of RNA silencing in plants. Similarly, RNAi has been shown to be induced by virus replication in animals. The antiviral capacity of RNA silencing has been used as a tool to generate virus resistance in plants. RNA-mediated virus resistance was obtained by expression of untranslatable viral coat-protein RNAs in transgenic plants. Expression of hairpin RNAs corresponding to viral sequences induced virus resistance in almost 100% of the transgenic plants. In analogy with RNA-mediated virus resistance in plants, RNAi technology is currently being used to inhibit viral replication in animal cells. Promising results have been obtained with RNAi against several animal viruses, both in in vitro and in vivo settings. For instance, Das et al. have demonstrated that inhibition of HIV-1 replication through RNAi is possible in stably transduced cells. However, HIV-1 escape mutants that were no longer inhibited appeared after several weeks of culture (Das et al., Journal of Virology, Vol. 78, No. 5 (2004) pages 2601-2605). Moreover, Gitlin et al. have shown that pre-treatment of human and mouse cells with double-stranded short interfering RNAs to the poliovirus genome markedly reduces the titer of virus progeny and promotes clearance of the virus from most of the infected cells. Protection is the result of direct targeting of the viral genome by siRNA. Also, in this study, escape variants appeared (Gitlin et al., Nature, Vol. 418 (2002), pages 430-434).

The present invention provides a way of improving RNAi-based treatment of an infection. With a method of the invention, a disease involving the presence of an organism is prevented and/or treated. A second generation of interfering RNAs is provided that is capable of counteracting escape mutants. Hence, long-term suppression of a mutating organism has become possible. In one preferred embodiment, a nucleic acid molecule of the present invention, therefore, comprises small interfering RNA.

A host cell is, for instance, provided with double-stranded RNA by transfection of double-stranded RNA molecules such as siRNA. siRNAs are, for instance, chemically or enzymatically synthesized. If long-term suppression of an organism is required, it is preferred to provide a cell with an expression cassette so that a double-stranded RNA is produced by the host cell. Intracellular synthesis of an siRNA is, for instance, achieved by expression of separate sense and antisense RNA fragments, which together form a dsRNA. Preferably, however, a short hairpin RNA (shRNA) is expressed. A short hairpin RNA comprises at least one base-paired stem (also called a base-paired duplex) comprising the sense and antisense strand of a siRNA, preferably with a length of between 15 and 40 nucleotides, more preferably with a length of between 15 and 30 nucleotides, more preferably with a length of between 19 and 25 nucleotides, most preferably with a length of between 21 and 23 nucleotides. The sense and antisense strand of an shRNA are linked with a small loop.

If a single short hairpin is used, the sense and antisense strands are joined through a single-stranded loop of several nucleotides. In one embodiment, multiple shRNA expression cassettes are inserted into a single vector. The units are clustered (parallel or antiparallel orientation) or inserted at different positions of the vector. In a preferred embodiment, multiple short hairpin RNAs are combined in a single expression construct. For instance, a stacked multiple shRNA structure is used, wherein the dsRNA sequences are separated by bulges, or a branched, tRNA-like multiple shRNA structure is used, wherein each branch is a separate shRNA. The efficiency of multiple shRNA constructs is preferably optimized by inclusion of signals capable of determining the intracellular location and stability of the RNA transcript. A multi-shRNA transcript is preferably made from a polymerase II or a polymerase III promoter. Examples of multi-shRNA constructs are shown in FIG. 3 and in Anderson et al., Oligonucleotides 13:303-312 (2003).

A short hairpin RNA is recognized by the DICER enzyme. Processing of a functional shRNA by DICER yields a siRNA of which the antisense strand is transferred to RISC, finally resulting in RNAi. FIG. 1 provides a schematic overview of RNAi interference with shRNA. Activation of the RNAi machinery by intracellular expression, preferably expression of a shRNA, has the advantage over transfection of synthetic siRNA that there is a constitutive transcription ensuring a basal level of RNAi activity. One aspect of the invention, therefore, provides a nucleic acid molecule of the invention comprising short hairpin RNA. In one embodiment, shRNA comprises a single short hairpin. In another embodiment, shRNA comprises a multiple short hairpin structure. A single shRNA of the invention preferably comprises a loop with a length of about two to 15 nucleotides. More preferably, the loop has a length of about three to ten nucleotides, most preferably about five to eight nucleotides. In one embodiment, the loop comprises hairpin-stabilizing tetraloop sequences. The base-paired duplex of an extended shRNA of the invention is furthermore preferably destabilized. This is, for instance, performed by inclusion of weak G-U base pairs and/or mispairs, and/or by destabilizing bulge or internal loop elements by modification of the sense strand.

Whereas the standard expression strategy for a shRNA transcript is the use of a Polymerase III (Pol III) promoter (e.g., H1 or U6), the expression of a longer transcript encoding multiple shRNAs is preferably optimized. This, for instance, includes the use of a Polymerase II (Pol II) promoter or the T7 expression system for cytoplasmic RNA expression. A promoter used in a short hairpin construct of the present invention is, for instance, constitutively expressed, either in all cell types/tissues or in a cell/tissue-specific manner. Preferably, however, the promoter is inducibly active so that the amount of expression of double-stranded RNA is regulated. A variety of viral vector systems is suitable for providing a host cell with a nucleic acid molecule of the invention. Preferably, a lentiviral and/or retroviral vector is used.

A method of the invention preferably involves targeting of an essential, conserved region so that an organism is limited in its capacity of circumventing treatment via escape mutants. In a preferred embodiment at least one genomic region of an organism is targeted that executes multiple functions. For instance, a region with an overlapping reading frame or even a triple overlap is targeted. An example of such region is the Tat-Rev-Env region in HIV. In one embodiment, a region comprising both a coding sequence and a regulatory sequence is targeted. Such region, for instance, comprises a sequence encoding a protein and an RNA/DNA replication signal such as a splicing signal or a sequence motif that controls RNA packaging or reverse transcription. If such regions are targeted, escape mutants are more difficult to arise because all functions need to be retained. A mutation that maintains one function may impair another function to such extent that the mutant is not viable. In view of these restrictions, the number of possible escape mutants is limited. Hence, if each possible escape mutant is to be targeted by a nucleic acid molecule or binding molecule of the invention, a limited number of nucleic acid molecules or binding molecules of the invention is needed. Mutations that would result in an escape mutant retaining all essential functions are readily computed or determined experimentally as, for instance, illustrated by Example 2 and FIGS. 8 and 9. One aspect of the invention, therefore, provides a nucleic acid molecule of the invention wherein the genomic nucleic acid sequence of the organism comprises at least one coding sequence and/or at least one DNA/RNA regulatory sequence.

Another aspect provides a nucleic acid molecule of the invention comprising a sequence that is complementary to a mutant comprising a coding sequence of an organism, wherein a nucleotide of a codon is substituted such that the resulting codon encodes the same amino acid residue. As illustrated by Example 2 and FIG. 8, possible escape mutants are readily computed.

In another embodiment, a nucleic acid molecule of the invention is provided, wherein the mutant comprises a genomic nucleic acid sequence of the organism with at least one mutation resulting in a significantly altered structure of the genomic nucleic acid sequence. The present inventors have identified a novel resistance mechanism through the formation of a stable nucleic acid structure by at least one mutation in or near the targeted sequence. Such mutants comprising an altered nucleic acid structure are computed using modeling programs known in the art such as computer-assisted RNA structure prediction programs. Alternatively, they are detected using long-term cultures in the presence of a (candidate) drug, such as, for instance, an antiviral. A nucleic acid molecule or binding molecule of the present invention is capable of binding such altered nucleic acid structure.

A nucleic acid molecule of the invention preferably comprises an inducible repressor and/or activator. In that case, the amount of expression and, therefore, the degree of treatment, is regulated. For instance, nucleic acid molecules complementary to all possible escape mutants are administrated. Expression is repressed until a certain escape mutant has evolved. Upon detection of such escape mutant, expression is enhanced by deactivating a repressor and/or activating an activator. In another embodiment, expression of a nucleic acid molecule of the invention is activated during treatment in order to prevent viral escape. In one embodiment, expression of a nucleic acid molecule of the invention is induced or repressed depending on possible side effects of a treatment and/or general health state of a patient.

A nucleic acid molecule and/or a binding molecule of the present invention is suitable for treating or preventing a disease involving the presence of an organism. One aspect of the invention, therefore, provides a nucleic acid molecule and/or binding molecule of the invention for use as a medicament. A nucleic acid molecule and/or binding molecule of the invention is particularly suitable for the preparation of a medicament or a vaccine against a disease involving an organism capable of mutating its genome. The organism preferably comprises HIV, Hepatitis B, Hepatitis C, SARS, Ebola, Influenza, West Nile and/or polio virus because these organisms have a high mutation rate.

A pharmaceutical composition comprising a nucleic acid molecule and/or binding molecule of the invention and a suitable diluent or carrier is also herewith provided. In one embodiment, the pharmaceutical composition comprises a vaccine.

Treatment of a (pathogenic) organism is particularly effective if a wild-type form of the organism is counteracted as well. One aspect of the invention, therefore, provides a combination therapy, wherein at least one compound capable of counteracting a wild-type organism is combined with a nucleic acid molecule and/or binding molecule of the present invention. Many compounds capable of counteracting a wild-type organism are known in the art. In this embodiment, at least one of these compounds is combined with a nucleic acid molecule or a binding molecule of the invention. The compound capable of counteracting a wild-type organism preferably targets at least one wild-type nucleic acid sequence of the organism. The compound preferably comprises double-stranded RNA, such as siRNA and/or shRNA, in order to use RNA interference. A compound capable of targeting a wild-type sequence is preferably combined with a nucleic acid molecule or binding molecule of the invention capable of specifically binding a mutant of the same wild-type sequence. In one embodiment, a second generation shRNA of the invention is combined with a primary shRNA capable of targeting a wild-type sequence. Alternatively, another kind of drug therapy is combined with a nucleic acid molecule or binding molecule of the invention. For instance, a currently used drug such as, for instance, an antiviral is combined with a nucleic acid molecule or binding molecule of the invention capable of specifically binding an escape mutant that is resistant to the drug. In this way, variants of an organism that are resistant to the drug are targeted by the nucleic acid molecule and/or binding molecule of the invention while variants resistant to the nucleic acid molecule and/or binding molecule of the invention are targeted by the drug. Various drug-resistance mutations are well known in the art. For instance, HIV drug-resistance mutations are included in the listing of the International AIDS Society (IAS) to help physicians classify the level of drug resistance. A nucleic acid molecule or binding molecule of the invention is preferably capable of specifically binding at least one of the drug-resistance mutants.

In another embodiment, frequently used CTL-escape or antibody escape routes of mutating organisms are blocked by a nucleic acid molecule and/or binding molecule of the invention anticipating such changes in the organism's genome.

A compound capable of counteracting a wild-type organism is preferably combined with a nucleic acid molecule and/or binding molecule of the invention in one pharmaceutical composition. In one embodiment, the compound capable of counteracting a wild-type organism and a nucleic acid molecule and/or binding molecule of the invention are administered at the same time. Alternatively, a compound capable of counteracting a wild-type organism and a nucleic acid molecule and/or binding molecule of the invention are administered at different time points. For instance, a nucleic acid molecule and/or binding molecule of the invention is administered once an escape mutant has evolved.

One aspect of the invention, therefore, provides a pharmaceutical composition comprising a nucleic acid molecule comprising a sequence that is complementary to a mutant of a genomic nucleic acid sequence of an organism capable of mutating its genome, and a nucleic acid molecule comprising a sequence that is complementary to a genomic nucleic acid sequence of the organism. Preferably, the nucleic acid molecule of the invention and the second nucleic acid molecule complementary to a genomic nucleic acid sequence are both complementary to (a mutant of) the same nucleic acid region of the genome. A pharmaceutical composition of the invention comprising a nucleic acid molecule of the invention, or binding molecule of the invention, and another kind of drug capable of at least in part counteracting the disease is also herewith provided. The other kind of drug, for instance, comprises a known antiviral drug.

Efficiency of treatment is enhanced when various nucleic acid sequences of an organism, or gene products thereof, are targeted because a mutant comprising a mutation in one nucleic acid sequence will still be targeted by a compound capable of targeting a second nucleic acid sequence of the organism. Escape mutants comprising a mutation in various nucleic acid sequences are more difficult to evolve as compared to escape mutants comprising a mutation in just one nucleic acid sequence. If escape mutants comprising a mutation in various nucleic acid sequences evolve, they are treated with a nucleic acid molecule or binding molecule of the invention. Hence, a preferred embodiment comprises a pharmaceutical composition comprising nucleic acid sequences that are complementary to mutants of at least two genomic nucleic acid sequences of the organism. More preferably, the pharmaceutical composition comprises nucleic acid sequences that are complementary to mutants of at least three genomic nucleic acid sequences of the organism. In one preferred embodiment, the three genomic nucleic acid sequences of the organism preferably comprise a Gag and Pol region of HIV. More preferably, the pharmaceutical composition comprises nucleic acid sequences that are complementary to mutants of at least four genomic nucleic acid sequences of the organism. Most preferably, a pharmaceutical composition is provided comprising nucleic acid sequences that are complementary to mutants of at least six genomic nucleic acid sequences of the organism. The higher the number of genomic nucleic acid sequences that, once they are mutated, are targeted by nucleic acid molecules and/or binding molecules of the invention, the higher the percentage of inhibition.

A nucleic acid sequence of the invention is administered in various ways known in the art. For instance, siRNAs are orally administered or injected. siRNAs are preferably administered with a suitable diluent or carrier such as, for instance, a physiologic salt solution. Preferably, cells are transduced with an expression cassette, preferably a shRNA construct. A gene delivery vehicle such as a viral vector system, preferably a lentiviral and/or retroviral vector, is particularly suitable. Methods of generating and using a gene delivery vehicle are well known in the art. Preferably, vectors and protocols according to (Dull et al., Journal of Virology 72, 8463-8471, 1998, and/or Seppen et al., Journal of Hepatology, April; 36(4); 459-65, 2002) are used. A gene delivery vehicle comprising a nucleic acid sequence of the invention is, therefore, also herewith provided. The gene delivery vehicle is suitable for ex vivo and in vivo gene therapy. Dose ranges of nucleic acids, gene delivery vehicles, binding molecules and/or other molecules according to the invention to be used in the therapeutical applications as described herein are designed on the basis of well-known rising dose studies in the clinic in clinical trials for which rigorous protocol requirements exist and which do not need further explanation.

A method of the invention is particularly suitable for counteracting an organism. Preferably, the organism is counteracted in vivo, comprising administration of a nucleic acid molecule or binding molecule of the invention to an individual suffering from, or at risk of suffering from, infection by the organism. In vitro applications comprise counteracting contaminants in (cell) culture. The invention thus provides a method for at least in part counteracting an organism, comprising providing the organism with a nucleic acid molecule or binding molecule of the invention. The organism is preferably additionally provided with a nucleic acid molecule comprising a sequence that is complementary to a genomic nucleic acid sequence of the organism. In that case, both wild-type forms and escape mutants are targeted. Targeting of multiple genomic nucleic acid sequences enhances the efficiency of a method of the invention. A preferred embodiment, therefore, provides a method of the invention, wherein the organism is provided with nucleic acid sequences that are complementary to mutants of at least two, more preferably at least three, even more preferably at least four, and most preferably at least six genomic nucleic acid sequences of the organism. In one preferred embodiment, at least two genomic nucleic acid sequences of the organism preferably comprise a Gag and Pol region of HIV.

A nucleic acid of the invention is suitable for interfering with a cell comprising an organism capable of mutating its genome, and/or with a cell comprising a nucleic acid sequence of an organism capable of mutating its genome. One embodiment of the invention, therefore, provides a method for interfering with a cell comprising an organism capable of mutating its genome and/or comprising a nucleic acid sequence of an organism capable of mutating its genome, comprising providing the cell with a nucleic acid sequence of the invention.

A method of the invention is preferably used against an organism involved with disease. Administration of a nucleic acid molecule and/or a binding molecule of the invention is capable of at least in part treating or preventing the disease during prolonged periods. The invention thus provides a method for at least in part treating or preventing a disease involved with an organism capable of mutating its genome, comprising providing an individual suffering from, or at risk of suffering from, the disease with a pharmaceutical composition of the invention. Preferably, the individual is additionally provided with another kind of drug capable of at least in part counteracting the organism.

A kit of parts comprising a nucleic acid molecule and/or binding molecule of the invention and another kind of drug capable of at least in part counteracting an organism is also herewith provided.

### DESCRIPTION OF THE DRAWINGS

FIG. 1: RNA interference with shRNA.

FIG. 2: Anticipating and countering viral escape with a second generation shRNA.

FIG. 3: RNAi induced by different RNA structures.

FIG. 4: Regions in the HIV-1 RNA genome that are more than 75% conserved among different viral isolates.

FIGS. 5A and 5B: shRNA's against conserved regions of HIV-1. Shown here is the relative CA-p24 production, relative against CA-p24 of virus production in the absence of RNAi-induced inhibition. CA-p24 was corrected for transfection efficiency by including Renilla in the transfection assay. shRNA expression plasmids (20 ng) and pLAI (100 ng), a molecular clone of HIV-1, on 2×10e4 293T cells in a 96-well format. About one in four shRNA's show a moderate to strong inhibition covering ten target regions. FIG. 5A: NEF 6-9 are the respective NEF 6-9f targets from Table 2D.

FIG. 6: Escape mutants are much less inhibited by shNEF. Shown here is the relative CA-p24 production, a measure for virus production corrected for transfection efficiency with renilla luciferase, after transfection of pLAI, the R3 or R3′ escape mutant with or without the shNEF expression plasmid. The point mutations in the target sequences of R3 and R3′ are bold and underlined. The escape mutants are much less inhibited by the shNEF. Since RNAi is highly sequence specific, the inhibition left is probably due to translational silencing; alternatively there could be some low level of RNAi still active.

FIG. 7: Proof of concept: Second generation shRNAs can block escape mutants. Shown here is the relative CA-p24 production, a measure for virus production corrected for transfection efficiency with renilla luciferase, after transfection of pLAI, the R3 or R3′ escape mutant with or without the complementary shRNA: shNEF, shR3 or shR3′, respectively. The point mutations in the target sequences of R3 and R3′ are bold and underlined. Inhibition is restored to levels obtained with the shNEF and pLAI when complementary shRNAs are used against the escape mutants.

FIG. 8: The Rev1/Tat5 target of shRNA R/T5. Shown here is the 19 nucleotide target of shRNA R/T4. For each reading frame, the nucleotide sequence, codons and amino acid sequence is shown. There are two silent mutations possible for this target, a A 6 C or a A18C mutation.

FIG. 9: The Pol/Vif target of shRNA P/4. Shown here is the 19 nucleotide target of shRNA P/4. For each reading frame, the nucleotide sequence, codons and amino acid sequence is shown. There are no silent mutations possible for this target, only mutations that result in at least one amino acid change of one of the reading frames.

FIG. 10: Validation of large shRNA screen. A subset from the large screen was co-transfected in a dilution series with pLAI. The data are not corrected for transfection; shown are the CA-p24 data. As a positive control, the shRNA against the Nef gene (Das et al., 2003) was used; as a negative control, a shRNA targeting the firefly luciferase gene or bluescript vector was used. The division in A, functional, B, intermediate and C, non-functional, was based on the large screen, averages for each group are shown in D.

FIG. 11: A lentiviral vector containing a single shRNA expression cassette.

FIG. 12: Virus replication in stably transduced SupT1 cells.

FIG. 13: A lentiviral vector containing a triple shRNA expression cassette, JS3-shRNA3.

FIG. 14: Co-transfection of pLuc-Gag-5 with lentiviral vector constructs.

FIG. 15: Co-transfection of luciferase reporter constructs Gag-5, Pol-1, Pol-47, and Pol-6.

FIG. 16: Transfection of pLAI clones with zero, one, or three shRNA expression cassettes.

FIG. 17: Schematic diagram of lentiviral vector production.

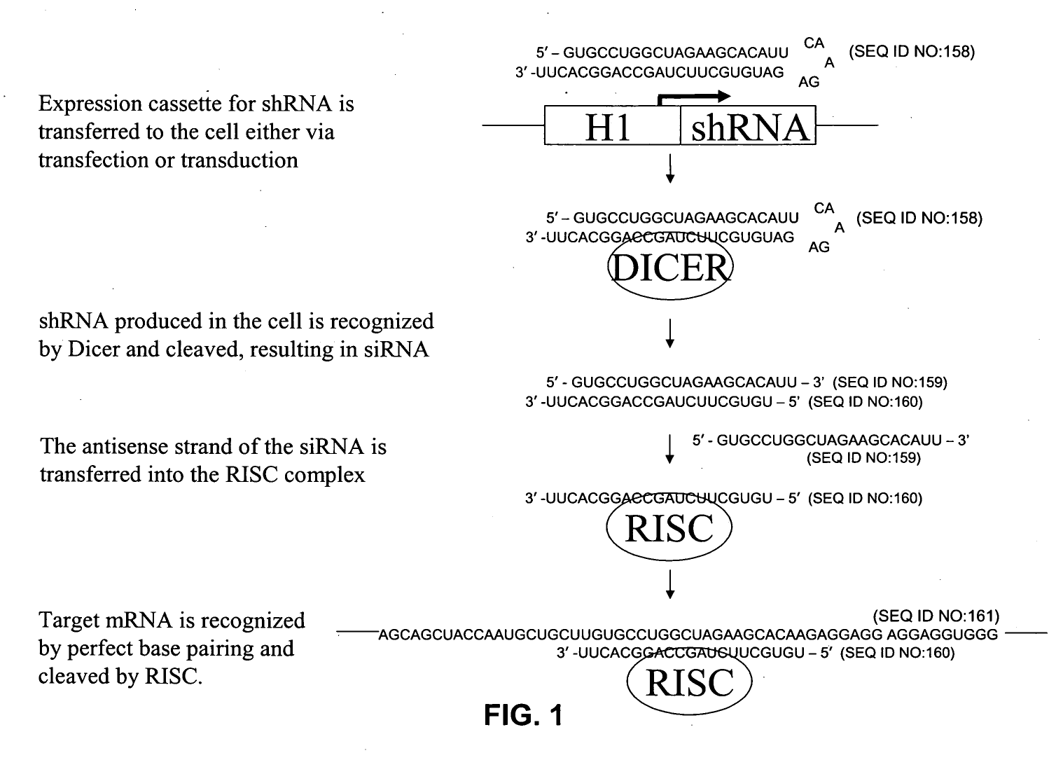

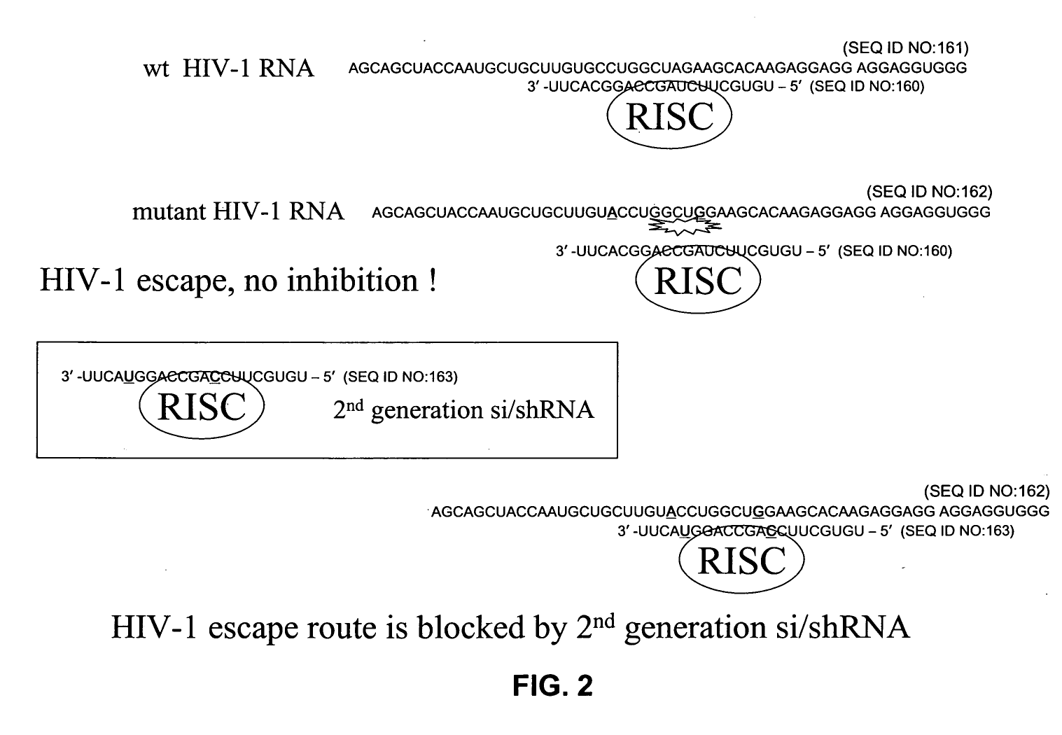

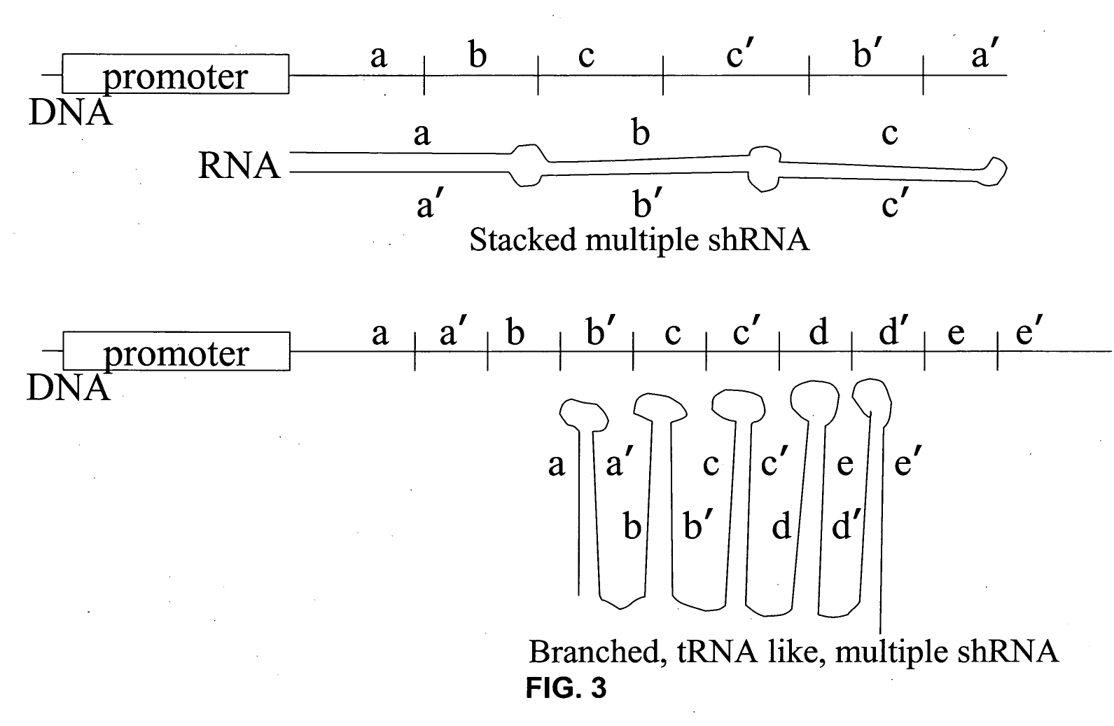

### DETAILED DESCRIPTION OF THE INVENTION

A method of the invention is particularly suitable for preventing and/or treating a virus-associated disease. Viruses such as HIV, polio, hepatitis, SARS, Ebola, influenza and/or West Nile virus are capable of escaping antiviral therapy because of their high mutation rate.

In view of this, current therapy directed against such mutating viruses often involves a combination of approaches. For instance, current therapy against HIV, sometimes called HAART (Highly Active Anti-Retroviral Therapy), often involves a combination of a nucleoside/tide reverse transcriptase inhibitor with a protease inhibitor and/or a non-nucleoside reverse transcriptase inhibitor.

In one embodiment, a method of the invention is used in order to counteract HIV. In that case, mature CD4+ T cells and/or CD34+ bone marrow precursor cells are preferably provided with a nucleic acid molecule or a binding molecule of the present invention. Preferably, mature CD4+ T cells and/or CD34+ bone marrow precursor cells are provided with a shRNA of the present invention. A variety of viral vector systems is suitable for providing a host cell with a nucleic acid molecule of the invention. Preferably, a lentiviral and/or retroviral vector is used. In one embodiment, an inducible promoter is used. For instance, during treatment of HIV, the Tet-system for doxycycline- (dox-) induced expression as disclosed in WO 01/20013 is preferred, or the HIV-1 LTR promoter that is activated only in HIV-infected cells by the viral Tat protein (Berkhout et al., Cell 1989; 59:273-282). In one embodiment, cytoplasmic expression using the T7 polymerase system is used for expression of shRNA, preferably multiple shRNA constructs. In one embodiment, a conditionally replicating HIV-based vector system is used as disclosed in WO 01/20013. This dox-dependent virus, which has been developed as a safe live-attenuated vaccine strain, is also suitable for use as a therapeutic virus by insertion of a nucleic acid molecule of the present invention, such as a shRNA expression cassette of the invention.

Hematopoietic stem cells are preferably transduced with an RNAi expression cassette of the present invention. These stem cells proliferate into mature T cells that resist HIV-1 infection. The virus-resistant cell population becomes enriched in case the non-resistant cells are infected and killed, either directly by the virus or indirectly by the immune system. The preferential survival of even a minority of siRNA-expressing cells results in their dominance over time.

Besides targeting of viral RNA, an alternative way to inhibit virus replication by RNAi is to silence the expression of cellular genes that are involved in viral replication. For HIV-1, these targets include the mRNAs encoding the CD4 receptor and the CCR5 or CXCR4 co-receptors. These receptors are essential for attachment of the HIV-1 particle to the cell and subsequent viral entry. RNAi against the viral RNA does not protect the cell against viral entry. By silencing the receptors, the HIV-1 particle will be unable to attach to and enter the cell, yielding a form of HIV-1 resistance. In vivo suppression of CD4 or CXCR4 is not preferred because of their immunological function. Therefore, preferably, expression of the co-receptor CCR5 is silenced. An inactivating mutation in the CCR5 gene is compatible with normal life, demonstrating that it plays no essential role in human physiology.

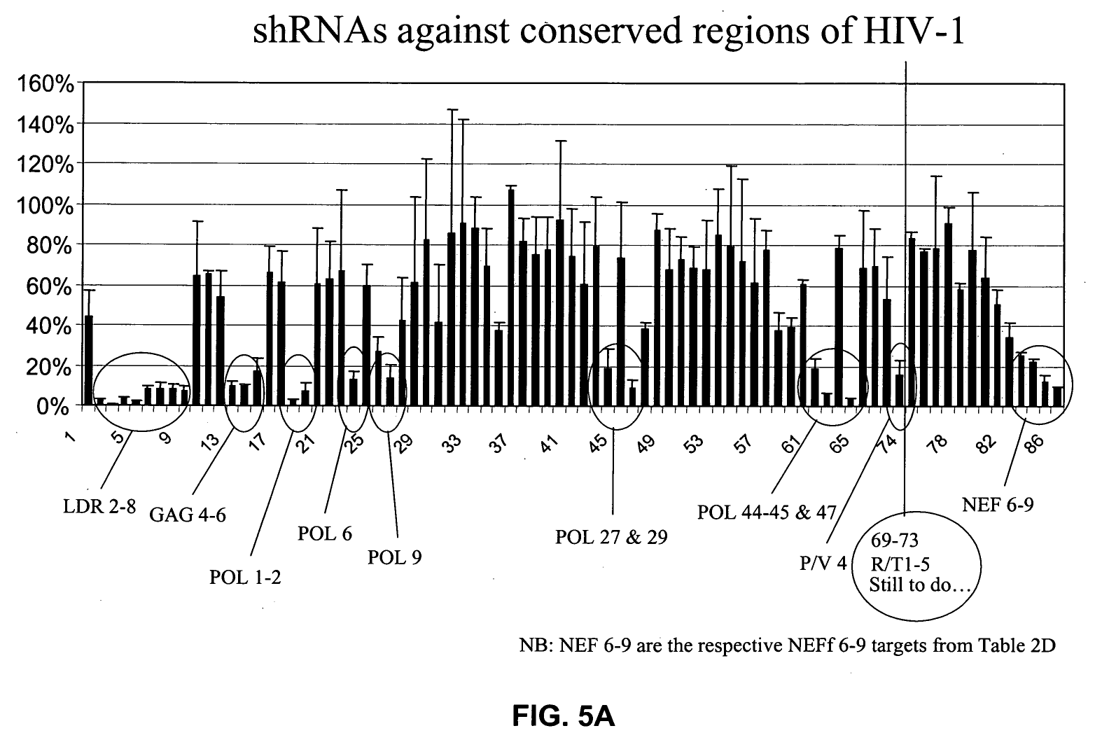

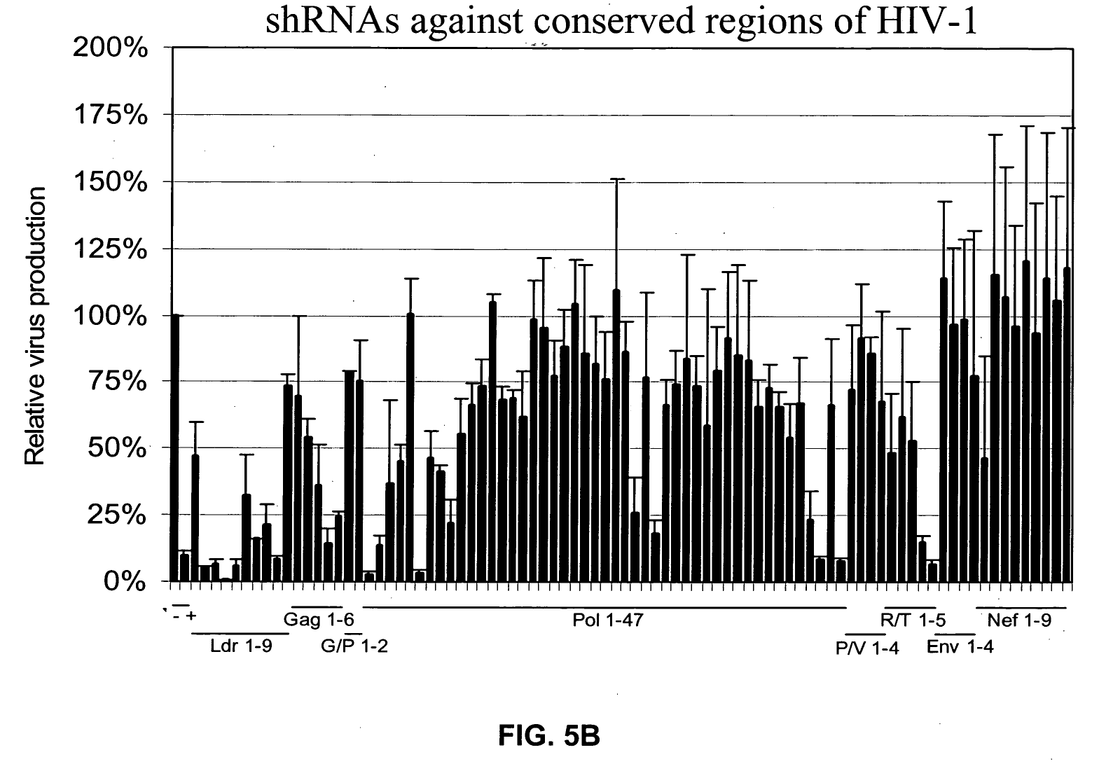

In a preferred embodiment, the invention furthermore provides eleven conserved regions of HIV-1 that are especially suitable for targeting during anti-HIV treatment. Although the HIV-1 genome comprises more conserved regions, it has been shown by the present inventors that the eleven conserved regions are particularly suitable for being targeted by nucleic acid silencing therapy. Silencing at least one region of the eleven conserved regions results in efficient virus inhibition. Furthermore, treatment is improved with a nucleic acid molecule or binding molecule of the invention capable of specifically binding a mutant of at least one of these eleven conserved regions. The eleven selected conserved regions are depicted in FIG. 5 and Table 1B.

In one aspect, the invention therefore provides a nucleic acid molecule comprising a sequence that is complementary to a conserved region as depicted in Table 1B, or to a functional part, derivative and/or analogue of the conserved region. Hence, the invention provides a nucleic acid molecule comprising a sequence that is complementary to at least part of at least one region selected from the group consisting of LDR 2-8, GAG 3-5, POL 1-2, POL 6, POL 9, POL 27, POL 29, POL 44-45, POL 47, PV 4 and R/T 4-5, the part preferably comprising at least ten, more preferably at least 15, even more preferably at least 17, even yet more preferably at least 18, and most preferably at least 19 nucleotides of the region, or that is complementary to a derivative or analogue of the region.

Most preferably, a nucleic acid molecule of the invention comprises a sequence that is complementary to at least part of LDR 2-8, GAG 3-5, POL 1-2, POL 6, POL 9, POL 27, POL 29, POL 44-45, POL 47 and/or R/T 4-5, the part preferably comprising at least ten, more preferably at least 15, even more preferably at least 17, even yet more preferably at least 18, and most preferably at least 19 nucleotides of the region, or that is complementary to a derivative or analogue of the region. Furthermore, a nucleic acid molecule is provided that is complementary to a mutant of a conserved region as depicted in Table 1B, or to a functional part or derivative of the mutant.

Hence, the invention provides a nucleic acid molecule comprising a sequence that is complementary to a mutant of at least part of at least one region selected from the group consisting of LDR 2-8, GAG 3-5, POL 1-2, POL 6, POL 9, POL 27, POL 29, POL 44-45, POL 47, P/V 4 and R/T 4-5, the part preferably comprising at least ten, more preferably at least 15, even more preferably at least 17, even yet more preferably at least 18, and most preferably at least 19 nucleotides of the region, or that is complementary to a derivative or analogue thereof. Most preferably, a nucleic acid molecule of the invention comprises a sequence that is complementary to a mutant of at least part of LDR 2-8, GAG 3-5, POL 1-2, POL 6, POL 9, POL 27, POL 29, POL 44-45, POL 47 and/or R/T 4-5, the part preferably comprising at least ten, more preferably at least 15, even more preferably at least 17, even yet more preferably at least 18, and most preferably at least 19 nucleotides of the region, or that is complementary to a derivative or analogue thereof.

The invention furthermore provides a NEF region of HIV-1 that is particularly suitable for targeting during anti-HIV treatment. According to the present invention, the NEF6f, NEF7f, NEF8f and NEF9f sequences depicted in Table 2D are particularly suitable for targeting during anti-HIV treatment. Hence, the invention provides a nucleic acid molecule comprising a sequence that is complementary to the NEF6f, NEF7f, NEF8f and NEF9f sequences depicted in Table 2D, the part preferably comprising at least ten, more preferably at least 15, even more preferably at least 17, even yet more preferably at least 18, and most preferably at least 19 nucleotides of the region, or that is complementary to a derivative or analogue thereof. Furthermore, a nucleic acid molecule is provided that is complementary to a mutant of the NEF6f, NEF7f, NEF8f and/or NEF9f sequence depicted in Table 2D.

Provided is, therefore, a nucleic acid molecule comprising a sequence that is complementary to a mutant of the NEF6f, NEF7f, NEF8f and/or NEF9f sequence depicted in Table 2D, the part preferably comprising at least ten, more preferably at least 15, even more preferably at least 17, even yet more preferably at least 18, and most preferably at least 19 nucleotides of the region, or that is complementary to a derivative or analogue thereof.

A functional part of a nucleic acid sequence is defined herein as a part of the nucleic acid, at least ten base pairs long, preferably at least 15 base pairs long, more preferably at least 19 base pairs long, comprising at least one characteristic (in kind, not necessarily in amount) as the nucleic acid sequence. Targeting of a part of a (conserved and/or essential) nucleic acid sequence of the invention by nucleic acid silencing therapy preferably results in inhibition of HIV. A “derivative of a nucleic acid sequence” is defined as a modified nucleic acid sequence comprising at least one characteristic (in kind, not necessarily in amount) as the nucleic acid sequence. The derivative, for instance, comprises the nucleic acid sequence with at least one silent mutation. A person skilled in the art is well capable of generating an analogue of a nucleic acid sequence. Such analogue, for instance, comprises a peptide nucleic acid (PNA), locked nucleic acid (LNA) and/or a ribozyme. Such analogue comprises at least one characteristic (in kind, not necessarily in amount) as the nucleic acid sequence.

The invention furthermore provides a nucleic acid molecule comprising a sequence that is complementary to a sequence as depicted in Tables 2A, 2B or 2C, or a functional part, derivative and/or analogue thereof. Such nucleic acid molecules are capable of inhibiting HIV and are, therefore, suitable for therapy. If at least one of such nucleic acid molecules is used during a prolonged period, escape mutants comprising mutations within these sequences, which are still capable of performing most, if not all, essential functions, are likely to arise. These escape mutants are targeted with a nucleic acid molecule or binding molecule of the present invention that is capable of specifically binding such escape mutant.

In one embodiment, the invention, therefore, provides a nucleic acid molecule comprising a sequence that is complementary to a mutant of a sequence as depicted in Tables 2A, 2B and/or 2C, or a functional part, derivative and/or analogue thereof. Escape mutants retaining their essential functions are computed as discussed above, or detected using long-term cultures in the presence of a nucleic acid sequence as depicted in Tables 2A, 2B and/or 2C.

A sequence as depicted in Tables 2A, 2B or 2C, or a functional part, derivative and/or analogue thereof, is capable of inhibiting HIV. Preferably, the percentage of HIV inhibition comprises at least 50% reduction in virus titer. A preferred embodiment, therefore, provides a nucleic acid molecule comprising a sequence that is complementary to a sequence as depicted in Tables 2A or 2B, or a functional part, derivative and/or analogue thereof. These sequences are capable of inhibiting HIV with a percentage of inhibition of at least 50% and are, therefore, particularly suitable for therapy.

Hence, the invention provides a nucleic acid molecule according to the invention comprising at least one sequence that is complementary to at least one sequence selected from the group consisting of AGGAGAGAGATGGGTGCGA (SEQ ID NO:1), GGAGAGAGATGGGTGCGAG (SEQ ID NO:2), GAGAGAGATGGGTGCGAGA (SEQ ID NO:3), AGAGAGATGGGTGCGAGAG (SEQ ID NO:4), GAGAGATGGGTGCGAGAGC (SEQ ID NO:5), AGAGATGGGTGCGAGAGCG (SEQ ID NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ ID NO:11), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ ID NO:17), GTGAAGGGGCAGTAGTAAT (SEQ ID NO:18), GGAAAACAGAUGGCAGGUG (SEQ ID NO:19), TATGGCAGGAAGAAGCGGA (SEQ ID NO:20), ATGGCAGGAAGAAGCGGAG (SEQ ID NO:21), AAGGAGAGAGATGGGTGCG (SEQ ID NO:22), TAGAAGAAATGATGACAGC (SEQ ID NO:23), TTAGCAGGAAGATGGCCAG (SEQ ID NO:24), TAGCAGGAAGATGGCCAGT (SEQ ID NO:25), ATTCCCTACAATCCCCAAA (SEQ ID NO:26), ACAATTTTAAAAGAAAAGG (SEQ ID NO:27), TACAGTGCAGGGGAAAGAA (SEQ ID NO:28), AAAATTCAAAATTTTCGGG (SEQ ID NO:29), GGAAAGGTGAAGGGGCAGT (SEQ ID NO:30), GAAAGGTGAAGGGGCAGTA (SEQ ID NO:31), AAGGTGAAGGGGCAGTAGT (SEQ ID NO:32) and CTTTTTAAAAGAAAAGGGG (SEQ ID NO:33).

Escape mutants arising as a result of selective pressure when treatment with a sequence as depicted in Tables 2A or 2B is used, are targeted with a nucleic acid molecule or binding molecule of the present invention that is capable of specifically binding such escape mutant. In one embodiment, the invention, therefore, provides a nucleic acid molecule comprising a sequence that is complementary to a mutant of a sequence as depicted in Tables 2A or 2B, or a functional part, derivative and/or analogue thereof. Hence, the invention provides a nucleic acid molecule according to the invention comprising at least one sequence that is complementary to a mutant of at least one sequence selected from the group consisting of AGGAGAGAGATGGGTGCGA (SEQ ID NO:1), GGAGAGAGATGGGTGCGAG (SEQ ID NO:2), GAGAGAGATGGGTGCGAGA (SEQ ID NO:3), AGAGAGATGGGTGCGAGAG (SEQ BD NO:4), GAGAGATGGGTGCGAGAGC (SEQ ID NO:5), AGAGATGGGTGCGAGAGCG (SEQ ID NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ BD NO:11), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ BD NO:17), GTGAAGGGGCAGTAGTAAT (SEQ ID NO:18), GGAAAACAGAUGGCAGGUG (SEQ ID NO:19), TATGGCAGGAAGAAGCGGA (SEQ ID NO:20), ATGGCAGGAAGAAGCGGAG (SEQ ID NO:21), AAGGAGAGAGATGGGTGCG (SEQ ID NO:22), TAGAAGAAATGATGACAGC (SEQ ID NO:23), TTAGCAGGAAGATGGCCAG (SEQ ID NO:24), TAGCAGGAAGATGGCCAGT (SEQ ID NO:25), ATTCCCTACAATCCCCAAA (SEQ ID NO:26), ACAATTTTAAAAGAAAAGG (SEQ ID NO:27), TACAGTGCAGGGGAAAGAA (SEQ ID NO:28), AAAATTCAAAATTTTCGGG (SEQ ID NO:29), GGAAAGGTGAAGGGGCAGT (SEQ ID NO:30), GAAAGGTGAAGGGGCAGTA (SEQ ID NO:31), AAGGTGAAGGGGCAGTAGT (SEQ ID NO:32) and CTTTTTAAAAGAAAAGGGG (SEQ ID NO:33).

Yet another preferred embodiment provides a nucleic acid molecule comprising a sequence that is complementary to a sequence as depicted in Table 2A, or a functional part, derivative and/or analogue thereof. These sequences are capable of inhibiting HIV with a percentage of inhibition of at least 80% and are, therefore, particularly suitable for therapy.

A preferred embodiment, therefore, provides a nucleic acid molecule comprising a sequence that is complementary to a sequence as depicted in Table 2A, or a functional part, derivative and/or analogue thereof. These sequences are capable of inhibiting HIV with a percentage of inhibition of at least 80% and are, therefore, particularly suitable for therapy.

Hence, the invention provides a nucleic acid molecule according to the invention comprising at least one sequence that is complementary to at least one sequence selected from the group consisting of AGGAGAGAGATGGGTGCGA (SEQ ID NO: 1), GGAGAGAGATGGGTGCGAG (SEQ ID NO:2), GAGAGAGATGGGTGCGAGA (SEQ ID NO:3), AGAGAGATGGGTGCGAGAG (SEQ ID NO:4), GAGAGATGGGTGCGAGAGC (SEQ ID NO:5), AGAGATGGGTGCGAGAGCG (SEQ ID NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ ID NO:11), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ ID NO:17), GTGAAGGGGCAGTAGTAAT (SEQ ID NO:18), GGAAAACAGAUGGCAGGUG (SEQ ID NO:19), TATGGCAGGAAGAAGCGGA (SEQ ID NO:20), and ATGGCAGGAAGAAGCGGAG (SEQ ID NO:21). The sequence most preferably comprises a sequence that is complementary to AGGAGAGAGATGGGTGCGA (SEQ ID NO:1), GGAGAGAGATGGGTGCGAG (SEQ ID NO:2), GAGAGAGATGGGTGCGAGA (SEQ ID NO:3), AGAGAGATGGGTGCGAGAG (SEQ ID NO:4), GAGAGATGGGTGCGAGAGC (SEQ ID NO:5), AGAGATGGGTGCGAGAGCG (SEQ ID NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ ID NO:1), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ ID NO:17), GTGAAGGGGCAGTAGTAAT (SEQ ID NO:18), TATGGCAGGAAGAAGCGGA (SEQ ID NO:20), and/or ATGGCAGGAAGAAGCGGAG (SEQ ID NO:21).

Escape mutants arising as a result of selective pressure when treatment with a sequence as depicted in Table 2A is used, are targeted with a nucleic acid molecule or binding molecule of the present invention that is capable of specifically binding such escape mutant. In one embodiment, the invention, therefore, provides a nucleic acid molecule comprising a sequence that is complementary to a mutant of a sequence as depicted in Table 2A, or a functional part, derivative and/or analogue thereof. Hence, the invention provides a nucleic acid molecule according to the invention comprising at least one sequence that is complementary to a mutant of at least one sequence selected from the group consisting of AGGAGAGAGATGGGTGCGA (SEQ ID NO:1), GGAGAGAGATGGGTGCGAG (SEQ ID NO:2), GAGAGAGATGGGTGCGAGA (SEQ ID NO:3), AGAGAGATGGGTGCGAGAG (SEQ ID NO:4), GAGAGATGGGTGCGAGAGC (SEQ ID NO:5), AGAGATGGGTGCGAGAGCG (SEQ ID NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ ID NO:1), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ ID NO:17), GTGAAGGGGCAGTAGTAAT (SEQ ID NO:18), GGAAAACAGAUGGCAGGUG (SEQ ID NO:19), TATGGCAGGAAGAAGCGGA (SEQ ID NO:20), and ATGGCAGGAAGAAGCGGAG (SEQ ID NO:21). The sequence most preferably comprises a sequence that is complementary to a mutant of AGGAGAGAGATGGGTGCGA (SEQ ID NO:1), GGAGAGAGATGGGTGCGAG (SEQ ID NO:2), GAGAGAGATGGGTGCGAGA (SEQ ID NO:3), AGAGAGATGGGTGCGAGAG (SEQ ID NO:4), GAGAGATGGGTGCGAGAGC (SEQ ID NO:5), AGAGATGGGTGCGAGAGCG (SEQ BD NO:6), GAGATGGGTGCGAGAGCGT (SEQ ID NO:7), AGATGGGTGCGAGAGCGTC (SEQ ID NO:8), AGAAGAAATGATGACAGCA (SEQ ID NO:9), GAAGAAATGATGACAGCAT (SEQ ID NO:10), AAGAAATGATGACAGCATG (SEQ ID NO:11), ACAGGAGCAGATGATACAG (SEQ ID NO:12), CAGGAGCAGATGATACAGT (SEQ ID NO:13), ATTGGAGGAAATGAACAAG (SEQ ID NO:14), TAGCAGGAAGATGGCCAGT (SEQ ID NO:15), CAGTGCAGGGGAAAGAATA (SEQ ID NO:16), AGGTGAAGGGGCAGTAGTA (SEQ ID NO:17), GTGAAGGGGCAGTAGTAAT (SEQ ID NO:18), TATGGCAGGAAGAAGCGGA (SEQ ID NO:20), and/or ATGGCAGGAAGAAGCGGAG (SEQ ID NO:21).

If a nucleic acid molecule according to the present invention comprises a sequence that is complementary to an HIV genomic nucleic acid sequence, it is particularly suitable for anti-AIDS therapy. For instance, gene therapy is used. In one embodiment, an RNAi-triggering nucleic acid sequence is transferred into an appropriate cell. Such a therapy is particularly suitable for the treatment of an individual that is chronically infected with HIV. In chronically infected individuals, HIV infects a significant fraction of the mature T cells each day, leading to cell killing directly by HIV and/or indirectly by the HIV-induced immune system. In addition, HIV infection involves the risk of inducing immune activation resulting in apoptosis in uninfected bystander cells. If a therapy according to the invention is applied, the preferential survival of cells expressing a nucleic acid sequence according to the invention will result in their outgrowth over time.

In one embodiment, an ex vivo gene therapy treatment of the patients mature CD4+ T cells from the blood and/or CD34+ hematopoietic stem cells from the bone marrow is performed, after which the CD4+ T cells and/or CD34+ hematopoietic stem cells are given back to the patient. The stem cells will proliferate into mature T cells and move to the periphery, thus forming a constant supply of cells that at least partially resist HIV infection. This means that even a relatively inefficient ex vivo gene therapy protocol is beneficial through partial reconstitution of the immune system.

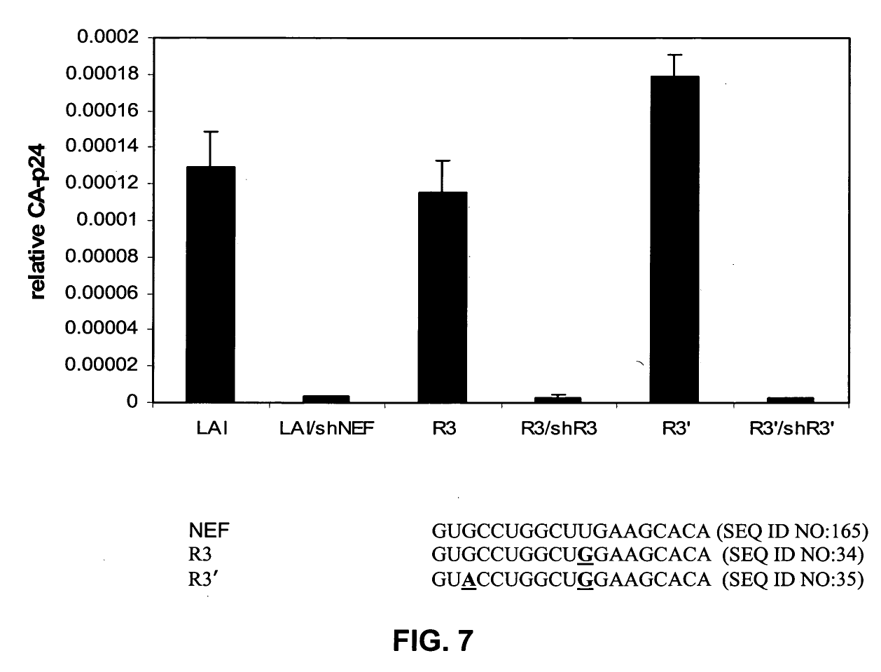

Two preferred sequences according to the invention, named R3 and R3′, are shown in FIG. 7. Administration of these sequences, preferably in the form of siRNA or shRNA, significantly improves inhibition of HIV. One aspect of the invention, therefore, provides a nucleic acid molecule that is complementary to the sequence GUGCCUGGCUGGAAGCACA (SEQ ID NO:34) and/or GUACCUGGCUGGAAGCACA (SEQ ID NO:35), or a functional part, derivative and/or analogue thereof.

The invention is further illustrated by the following examples. These examples do not limit the invention in any way.

### EXAMPLES

### Example 1

### Materials and Methods

Conserved HIV-1 sequences

20-mer sequences covering the complete genome of the HIV-1 molecular clone pLAI were aligned with all complete HIV-1 genome sequences available, 170 at that time, from the Los Alamos database. The LAI genome size is 9229 bases; this means that, in total, 9211 different 20-mer sequences are possible. We defined conserved sequences as target regions that have 100% identity with the 20-mer sequence in at least 75% of all HIV-1 genomes. The conserved regions with their nucleotide position, genomic region and sequence are shown in Table 1A.

### ShRNA-Expressing Plasmids

ShRNA-expressing plasmids were constructed as follows. The pSUPER vector, a gift from T. R. Brummelkamp, the Netherlands Cancer Institute (Brummelkamp et al., 2002), that contains the H1 promoter, was linearized with BgIII and HindIII. Forward and reverse strands of the oligos, which contain the shRNA-expressing sequence targeting the conserved regions, were annealed and cloned into the vector.

The sequence targeting the conserved region was 19 nt, that means for a stretch of 23 nucleotides, 5 different shRNAs were designed. The oligo sequences were as follows:

Forward primer:

(SEQ ID NO:36)

sense anti-sense
GATCCCCNNNNNNNNNNNNNNNNNNNTTCAAGAGANNNNNNNNNNNNNNN
NNNNTTTTTGGAAA
Reverse primer

(SEQ ID NO:37)

sense anti-
AGCTTTTCCAAAAANNNNNNNNNNNNNNNNNNNTCTCTTGAANNNNNNNN
sense
NNNNNNNNNNNGGG

The shRNA-expressing plasmids targeting the conserved regions of HIV-1 were numbered from 1 to 86 and were also numbered according to the genomic region, as is shown in Table 1A.

Co-Transfection of shRNA Constructs with pLAI

In a 96-well format, co-transfection experiments of the shRNA-vector and pLAI were performed. Per well, 2×10⁴ 293T cells were seeded in 200 μl DMEM with 10% FCS without antibiotics. The next day, 200 ng of pLAI, 20 ng of shRNA-vector and 0.63 ng of pRL (Renilla Luciferase) was transfected with 0.5 μl lipofectamine-2000 in a reaction volume of 50 μl according to the manufacturer's instructions. Eight hours post-transfection, the medium was replaced with 200 μl medium containing antibiotics. Forty-eight hours after transfection, medium samples were taken for CA-p24 ELISA and cells were lysed for Renilla measurements. CA-p24 is a measure of virus production. To correct for transfection variation, relative CA-24 values were determined by dividing the CA-p24 measurements by the Renilla values obtained. Negative controls that were included were pBS, pSUPER (without insert) and an empty control. Positive controls were shRNAs targeting the Nef gene (Das et al., 2003) and the Gag gene (Capodici et al., J. Immunol. 2002 Nov. 1; 169(9):5196-201).

Smaller scale transfections were carried out in a 24-well format. Per well, 1-1.5×10⁵ 293T cells were seeded in 500 μl DMEM with 10% FCS without antibiotics. The next day, 500 ng of pLAI, 0-500 ng of shRNA-vector and 2.5 ng of pRL (Renilla Luciferase) was transfected with 1-2 μl lipofectamine-2000 in a reaction volume of 100 μl according to the manufacturer's instructions. The remainder of the experiment was carried out as described above.

### Results

First, we identified conserved regions in the HIV-1 clone pLAI. Conservedness was defined as regions in LAI having 100% identity with at least 75% of all complete HIV-1 genomes available in the Los Alamos database at that time (170). Nineteen different regions were found (Table 1A; however, in this first screening, the NEF 1-9 sequence GAAAAGGGGGGACUGGAAGGGCUAAUU (SEQ ID NO:38) was used as the target sequence instead of the NEF 1-9 sequence depicted in Table 1A). For these regions, shRNA-expressing plasmids were constructed containing 19 nucleotides of the target sequences. For a stretch of 25 nucleotides, this means that seven different shRNAs were made. In co-transfection experiments, all constructs were tested for their ability to inhibit HIV-1 production (FIG. 5A, Table 2). We divided the shRNAs into different groups; strong shRNAs with 80-100% inhibition, intermediate shRNAs with 50-80% inhibition and weak inhibitors with 0-50% inhibition. Of the 81 shRNAs tested, 21 were strong, 13 were intermediate and 47 were weak. The results of this experiment were subsequently confirmed in a second experiment.

In this second experiment, shRNA-expressing plasmids containing 19 nucleotides of the R/T 1-5 region were also tested. In this second experiment, the NEF 1-9 sequence depicted in Table 1A was used as the target sequence. The results are shown in FIG. 5B. The second experiment confirms the results of the first experiment (with the exception of PV-4, which is probably due to human inaccuracy).

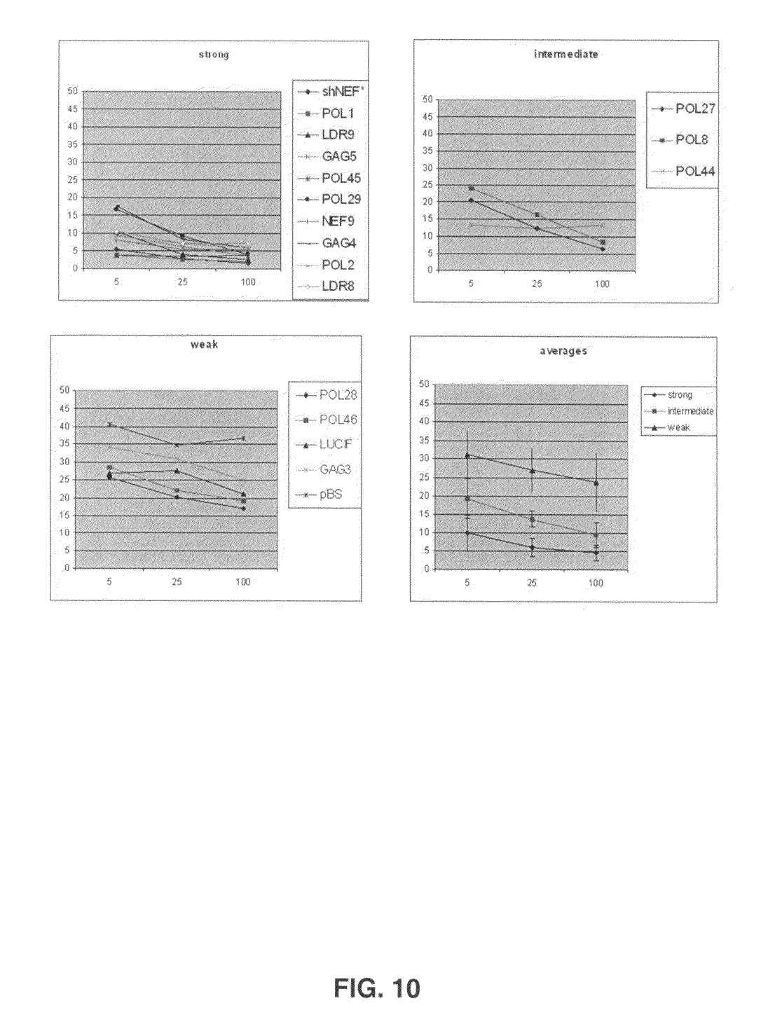

To confirm the results from the 96-well experiment, a selection was made of several shRNAs representing each group. These shRNAs were tested in a dilution series of 5, 25 and 100 ng, respectively (FIG. 10). From the individual dose-response curves as well as from the average dose-response curves, it can be concluded that these shRNAs fall into the different functional groups, validating the data obtained from the first screening of the 81 shRNAs.

TABLE 1A

Conserved regions in LAI
There are 19 different regions in the LAI genome
that are highly conserved among different viral
isolates. Shown here is the nucleotide position,
genomic region and sequence of these different
regions. shRNAs were designed to target these
regions. The numbers of the different shRNA-
expressing plasmids are shown, as well as the
name of the different shRNA constructs.

| | | | | | |
|---|---|---|---|---|---|
| | | | SEQ | | |
| | | | ID | | |
| start | target | sequence | NO: | nr | name |

| | | | | | |
|---|---|---|---|---|---|
| 326 | leader | AAGGAGAGAGAUGGGU | 39 | 1-9 | LDR 1-9 |
| | | GCGAGAGCGUC | | | |
| 1146 | gag | AAUAAAAUAGUAAGAA | 40 | 10-11 | GAG 1-2 |
| | | UGUA | | | |
| 1363 | gag | UAGAAGAAAUGAUGAC | 41 | 12-15 | GAG 3-6 |
| | | AGCAUG | | | |
| 1623 | gag/pol | CAGGCUAAUUUUUUAG | 42 | 16-17 | G/P 1-2 |
| | | GGAA | | | |
| 1910 | pol | ACAGGAGCAGAUGAUA | 43 | 18-19 | POL 1-2 |
| | | CAGU | | | |
| 1957 | pol | AUGGAAACCAAAAAUG | 44 | 20-22 | POL 3-5 |
| | | AUAGG | | | |
| 3755 | pol | AUUGGAGGAAAUGAAC | 45 | 23-24 | POL 6-7 |
| | | AAGU | | | |
| 4121 | pol | UUAGCAGGAAGAUGGC | 46 | 25-26 | POL 8-9 |
| | | CAGU | | | |
| 4232 | pol | AUUCCCUACAAUCCCC | 47 | 27-28 | POL 10-11 |
| | | AAAG | | | |
| 4358 | pol | CACAAUUUUAAAAGAA | 48 | 29-43 | POL 12-26 |
| | | AAGGGGGGAUUGGGG | | | |
| | | GG | | | |
| 4391 | pol | UACAGUGCAGGGGAAA | 49 | 44-46 | POL 27-29 |
| | | GAAUA | | | |
| 4466 | pol | AAAAUUCAAAAUUUUC | 50 | 47-48 | POL 30-31 |
| | | GGGU | | | |
| 4470 | pol | UUCAAAAUUUUCGGGU | 51 | 49-51 | POL 32-34 |
| | | UUAUU | | | |
| 4475 | pol | AAUUUUCGGGUUUAUU | 52 | 52-53 | POL 35-36 |
| | | ACAG | | | |
| 4535 | pol | CUCUGGAAAGGUGAAG | 53 | 54-64 | POL 37-47 |
| | | GGGCAGUAGUAAU | | | |
| 4622 | pol/vif | UAUGGAAAACAGAUGG | 54 | 65-68 | P/V 1-4 |
| | | CAGGUG | | | |
| 5547 | rev/tat | UCCUAUGGCAGGAAGA | 55 | 69-73 | R/T 1-5 |
| | | AGCGGAG | | | |
| 7391 | env | CAGCAGGAAGCACUAU | 56 | 74-77 | ENV 1-4 |
| | | GGGCGC | | | |
| 8656 | nef | CUUUUUAAAAGAAAAG | 57 | 78-86 | NEF 1-9 |
| | | GGGGGACUGGA | | | |

TABLE 1B

Conserved regions of HIV that are particularly
suitable for targeting during HIV treatment

| | | |
|---|---|---|
| SEQ ID NO: | Target sequence | Name |

| | | |
|---|---|---|
| 58 | AGGAGAGAGAUGGGUGCGAGAGCGU | LDR 2-8 |
| 59 | UAGAAGAAAUGAUGACAGCAU | GAG 3-5 |
| 43 | ACAGGAGCAGAUGAUACAGU | POL 1-2 |
| 60 | AUUGGAGGAAAUGAACAAG | POL 6 |
| 61 | UAGCAGGAAGAUGGCCAGU | POL 9 |
| 62 | UACAGUGCAGGGGAAAGAA | POL 27 |
| 63 | CAGUGCAGGGGAAAGAAUA | POL 29 |
| 64 | AAGGUGAAGGGGCAGUAGUA | POL 44-45 |
| 65 | GUGAAGGGGCAGUAGUAAU | POL 47 |
| 66 | GGAAAACAGAUGGCAGGUG | P/V 4 |
| 67 | UAUGGCAGGAAGAAGCGGAG | R/T 4-5 |

Tables 2A-2C shRNAs against conserved regions of HIV-1

Shown here is the percentage of inhibition for each shRNA and the sense sequence that is targeted by the shRNA. A division was made into three groups: strong, intermediate and weak shRNAs.

TABLE 2A
strong inhibition, 80-100%

| | | | |
|---|---|---|---|
| 2 | SEQ ID NO:68 | LDR2 | AGGAGAGAGAUGGGUGCGA |
| 3 | SEQ ID NO:69 | LDR3 | GGAGAGAGAUGGGUGCGAG |
| 4 | SEQ ID NO:70 | LDR4 | GAGAGAGAUGGGUGCGAGA |
| 5 | SEQ ID NO:71 | LDR5 | AGAGAGAUGGGUGCGAGAG |
| 6 | SEQ ID NO:72 | LDR6 | GAGAGAUGGGUGCGAGAGC |
| 7 | SEQ ID NO:73 | LDR7 | AGAGAUGGGUGCGAGAGCG |
| 8 | SEQ ID NO:74 | LDR8 | GAGAUGGGUGCGAGAGCGU |
| 9 | SEQ ID NO:75 | LDR9 | AGAUGGGUGCGAGAGCGUC |
| 13 | SEQ ID NO:76 | GAG4 | AGAAGAAAUGAUGACAGCA |
| 14 | SEQ ID NO:77 | GAG5 | GAAGAAAUGAUGACAGCAU |
| 15 | SEQ ID NO:78 | GAG6 | AAGAAAUGAUGACAGCAUG |
| 18 | SEQ ID NO:79 | POL1 | ACAGGAGCAGAUGAUACAG |
| 19 | SEQ ID NO:80 | POL2 | CAGGAGCAGAUGAUACAGU |
| 23 | SEQ ID NO:60 | POL6 | AUUGGAGGAAAUGAACAAG |
| 26 | SEQ ID NO:61 | POL9 | UAGCAGGAAGAUGGCCAGU |
| 46 | SEQ ID NO:63 | POL29 | CAGUGCAGGGGAAAGAAUA |
| 62 | SEQ ID NO:81 | POL45 | AGGUGAAGGGGCAGUAGUA |
| 64 | SEQ ID NO:65 | POL47 | GUGAAGGGGCAGUAGUAAU |
| 68 | SEQ ID NO:66 | P/V4 | GGAAAACAGAUGGCAGGUG |
| 72 | SEQ ID NO:82 | R/T4 | UAUGGCAGGAAGAAGCGGA |
| 73 | SEQ ID NO:83 | R/T5 | AUGGCAGGAAGAAGCGGAG |

TABLE 2B
intermediate inhibition, 50-80%

| | | | |
|---|---|---|---|
| 1 | SEQ ID NO:84 | LDR1 | AAGGAGAGAGAUGGGUGCG |
| 12 | SEQ ID NO:85 | GAG3 | UAGAAGAAAUGAUGACAGC |
| 25 | SEQ ID NO:86 | POL8 | UUAGCAGGAAGAUGGCCAG |
| 26 | SEQ ID NO:61 | POL9 | UAGCAGGAAGAUGGCCAGU |
| 27 | SEQ ID NO:87 | POL10 | AUUCCCUACAAUCCCCAAA |
| 30 | SEQ ID NO:88 | POL13 | ACAAUUUUAAAAGAAAAGG |
| 44 | SEQ ID NO:89 | POL27 | UACAGUGCAGGGGAAAGAA |
| 47 | SEQ ID NO:90 | POL30 | AAAAUUCAAAAUUUUCGGG |
| 58 | SEQ ID NO:91 | POL41 | GGAAAGGUGAAGGGGCAGU |
| 59 | SEQ ID NO:92 | POL42 | GAAAGGUGAAGGGGCAGUA |
| 61 | SEQ ID NO:93 | POL44 | AAGGUGAAGGGGCAGUAGU |
| 78 | SEQ ID NO:94 | NEF1 | CUUUUUAAAAGAAAAGGGG |

TABLE 2C
weak inhibition, 0-50%

| | | | |
|---|---|---|---|
| 10 | SEQ ID NO:95 | GAG1 | AAUAAAAUAGUAAGAAUGU |
| 11 | SEQ ID NO:96 | GAG2 | AUAAAAUAGUAAGAAUGUA |
| 16 | SEQ ID NO:97 | G/P1 | CAGGCUAAUUUUUUAGGGA |
| 17 | SEQ ID NO:98 | G/P2 | AGGCUAAUUUUUUAGGGAA |
| 20 | SEQ ID NO:99 | POL3 | AUGGAAACCAAAAAUGAUA |
| 21 | SEQ ID NO:100 | POL4 | UGGAAACCAAAAAUGAUAG |
| 22 | SEQ ID NO:101 | POL5 | GGAAACCAAAAAUGAUAGG |
| 24 | SEQ ID NO:102 | POL7 | UUGGAGGAAAUGAACAAGU |
| 28 | SEQ ID NO:103 | POL11 | UUCCCUACAAUCCCCAAAG |
| 29 | SEQ ID NO:104 | POL12 | CACAAUUUUAAAAGAAAAG |
| 31 | SEQ ID NO:105 | POL14 | CAAUUUUAAAAGAAAAGGG |
| 32 | SEQ ID NO:106 | POL15 | AAUUUUAAAAGAAAAGGGG |
| 33 | SEQ ID NO:107 | POL16 | AUUUUAAAAGAAAAGGGGG |
| 34 | SEQ ID NO:108 | POL17 | UUUUAAAAGAAAAGGGGGG |
| 35 | SEQ ID NO:109 | POL18 | UUUAAAAGAAAAGGGGGGA |
| 36 | SEQ ID NO:110 | POL19 | UUAAAAGAAAAGGGGGGAU |
| 37 | SEQ ID NO:111 | POL20 | UAAAAGAAAAGGGGGGAUU |
| 38 | SEQ ID NO:112 | POL21 | AAAAGAAAAGGGGGGAUUG |
| 39 | SEQ ID NO:113 | POL22 | AAAGAAAAGGGGGGAUUGG |
| 40 | SEQ ID NO:114 | POL23 | AAGAAAAGGGGGGAUUGGG |
| 41 | SEQ ID NO:115 | POL24 | AGAAAAGGGGGGAUUGGGG |
| 42 | SEQ ID NO:116 | POL25 | GAAAAGGGGGGAUUGGGGG |
| 43 | SEQ ID NO:117 | POL26 | AAAAGGGGGGAUUGGGGGG |
| 45 | SEQ ID NO:118 | POL28 | ACAGUGCAGGGGAAAGAAU |
| 48 | SEQ ID NO:119 | POL31 | AAAUUCAAAAUUUUCGGGU |
| 49 | SEQ ID NO:120 | POL32 | UUCAAAAUUUUCGGGUUUA |
| 50 | SEQ ID NO:121 | POL33 | UCAAAAUUUUCGGGUUUAU |
| 51 | SEQ ID NO:122 | POL34 | CAAAAUUUUCGGGUUUAUU |
| 52 | SEQ ID NO:123 | POL35 | AAUUUUCGGGUUUAUUACA |
| 53 | SEQ ID NO:124 | POL36 | AUUUUCGGGUUUAUUACAG |
| 54 | SEQ ID NO:125 | POL37 | CUCUGGAAAGGUGAAGGGG |
| 55 | SEQ ID NO:126 | POL38 | UCUGGAAAGGUGAAGGGGC |
| 56 | SEQ ID NO:127 | POL39 | CUGGAAAGGUGAAGGGGCA |
| 57 | SEQ ID NO:128 | POL40 | UGGAAAGGUGAAGGGGCAG |
| 60 | SEQ ID NO:129 | POL43 | AAAGGUGAAGGGGCAGUAG |
| 63 | SEQ ID NO:130 | POL46 | GGUGAAGGGGCAGUAGUAA |
| 65 | SEQ ID NO:131 | P/V1 | UAUGGAAAACAGAUGGCAG |
| 66 | SEQ ID NO:132 | P/V2 | AUGGAAAACAGAUGGCAGG |
| 67 | SEQ ID NO:133 | P/V3 | UGGAAAACAGAUGGCAGGU |
| 69 | SEQ ID NO:134 | R/T1 | UCCUAUGGCAGGAAGAAGC |
| 70 | SEQ ID NO:135 | R/T2 | CCUAUGGCAGGAAGAAGCG |
| 71 | SEQ ID NO:136 | R/T3 | CUAUGGCAGGAAGAAGCGG |
| 74 | SEQ ID NO:137 | ENV1 | CAGCAGGAAGCACUAUGGG |
| 75 | SEQ ID NO:138 | ENV2 | AGCAGGAAGCACUAUGGGC |
| 76 | SEQ ID NO:139 | ENV3 | GCAGGAAGCACUAUGGGCG |
| 77 | SEQ ID NO:140 | ENV4 | CAGGAAGCACUAUGGGCGC |
| 79 | SEQ ID NO:141 | NEF2 | UUUUUAAAAGAAAAGGGGG |
| 80 | SEQ ID NO:142 | NEF3 | UUUUAAAAGAAAAGGGGGG |
| 81 | SEQ ID NO:143 | NEF4 | UUUAAAAGAAAAGGGGGGA |
| 82 | SEQ ID NO:144 | NEF5 | UUAAAAGAAAAGGGGGGAC |
| 83 | SEQ ID NO:145 | NEF6 | UAAAAGAAAAGGGGGGACU |
| 84 | SEQ ID NO:146 | NEF7 | AAAAGAAAAGGGGGGACUG |
| 85 | SEQ ID NO:147 | NEF8 | AAAGAAAAGGGGGGACUGG |
| 86 | SEQ ID NO:148 | NEF9 | AAGAAAAGGGGGGACUGGA |

TABLE 2D

Tested targets in NEF that are not highly
conserved, NEF 6-9f inhibit HIV-1.

| | | |
|---|---|---|
| NEF1f | SEQ ID NO:149 | GAAAAGGGGGGACUGGAAG |
| NEF2f | SEQ ID NO:150 | AAAAGGGGGGACUGGAAGG |
| NEF3f | SEQ ID NO:151 | AAAGGGGGGACUGGAAGGG |
| NEF4f | SEQ ID NO:152 | AAGGGGGGACUGGAAGGGC |
| NEF5f | SEQ ID NO:153 | AGGGGGGACUGGAAGGGCU |
| NEF6f | SEQ ID NO:154 | GGGGGGACUGGAAGGGCUA |
| NEF7f | SEQ ID NO:155 | GGGGGACUGGAAGGGCUAA |
| NEF8f | SEQ ID NO:156 | GGGGACUGGAAGGGCUAAU |
| NEF9f | SEQ ID NO:157 | GGGACUGGAAGGGCUAAUU |

### Example 2

### Escape Mutants

Besides targeting conserved regions, the number of escape routes is limited by selecting (conserved) regions of the viral genome that execute multiple functions. This includes overlapping reading frames, but also sequences that encode both a protein and an important RNA/DNA sequence (e.g., a splicing signal or a sequence motif that controls RNA packaging or reverse transcription).

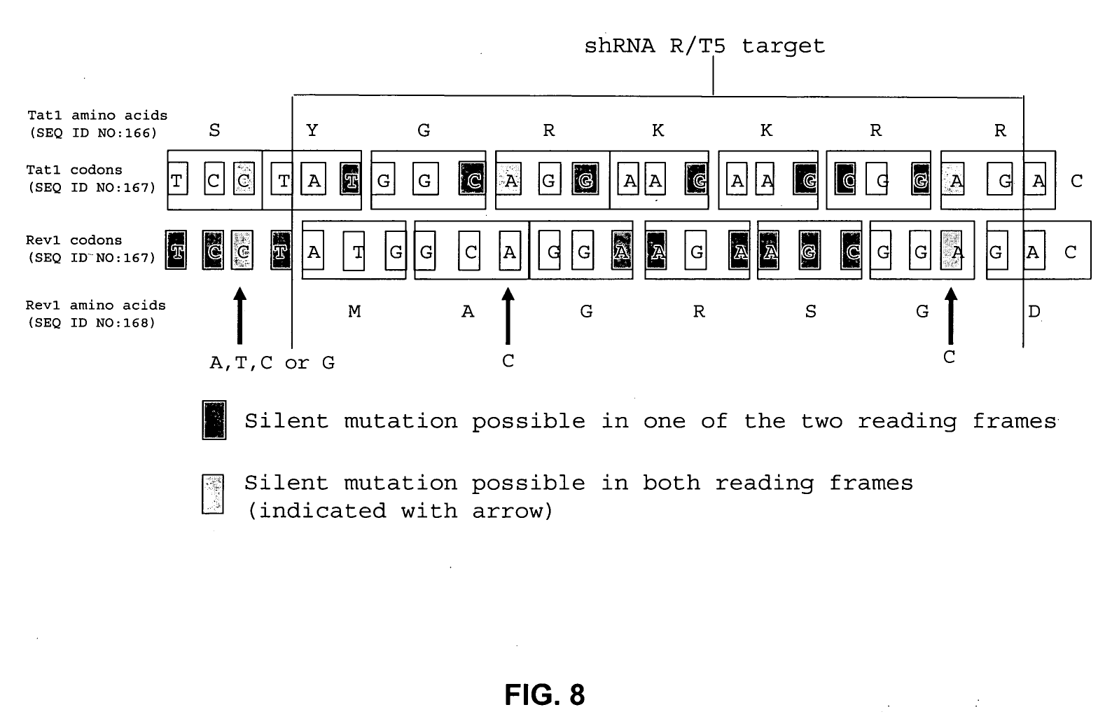

For example, the shRNA R/T5 targets the Tat1 reading frame and also the Rev1 reading frame. If we make the assumption that for both Tat and Rev the amino acid sequence is conserved, then only mutations are allowed that are silent in both reading frames. For the shRNA targeting R/T5, this is shown in FIG. 8. Two silent mutations can occur in the shRNA R/T5 target: A6C or A18C (NB nt 1 is start of target sequence). Therefore, in addition to the primary shRNA, shRNAs that are complementary to the two possible silent escape variants are preferably also included in anti-HIV treatment.

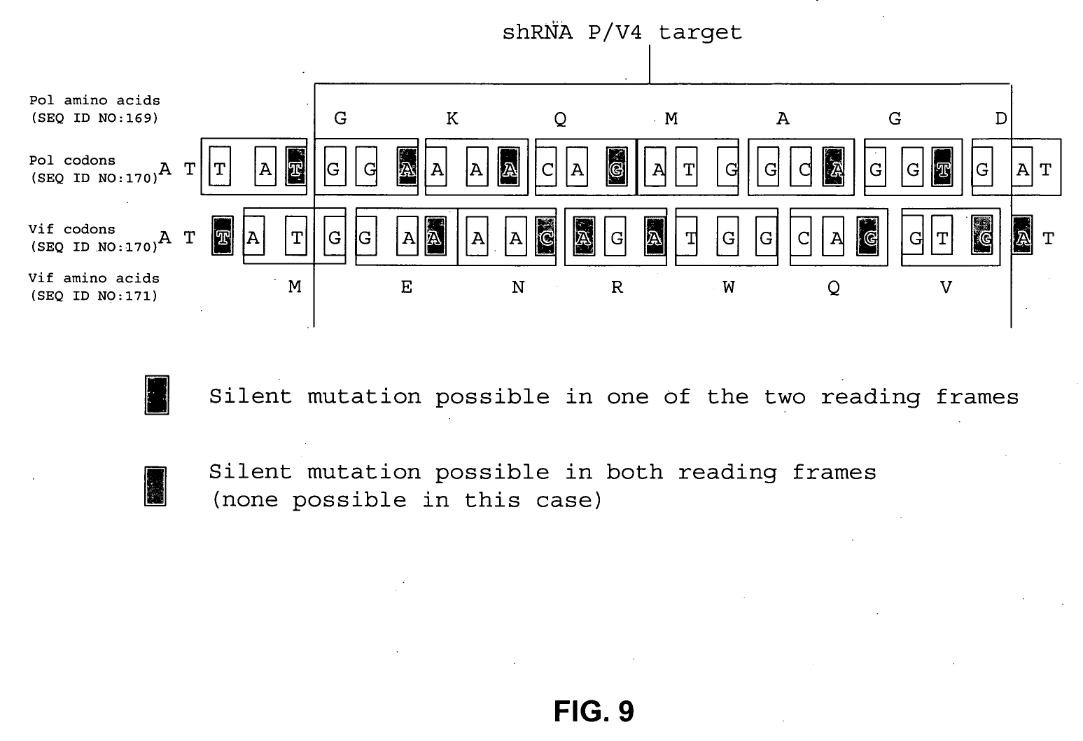

Another example is P/V4 target, which was shown to be effectively targeted by a shRNA. The P/V4 target sequence contains the Pol and the Vif reading frames, FIG. 9. When we again make the assumption that only silent mutations are allowed, there is no escape possible for this target, and no silent mutation can occur. Alternatively, based on protein structure, we predict what amino acid substitutions for each reading frame are allowed. For these substitutions, certain nucleic acid mutations are needed, again only the mutations that will result in both functional Pol and Vif proteins will be allowed, limiting the possibilities for escape. A second generation of shRNAs targeting these possible escape mutants are preferably combined with the primary shRNA.

For conserved regions of the HIV-1 genome that contain multiple regulatory RNA/DNA sequences and/or reading frames, similar exercises are performed. In each case, the possibilities of escape are limited due to the multiple functions of the target and are restricted to the few nucleotide positions for which variation is allowed. Only mutations are allowed that do not significantly impair any of its multiple functions. Primary shRNAs are preferably combined with the second generation to prevent escape.

Additionally, there is a preference for mismatches at certain positions. If we take a look at viral escape, and take into account the knowledge about the specificity of RNAi, it is striking that there is a preference in most virus escape mutants reported so far to have point mutations at the ninth nucleotide position of the target mRNA. Three out of six reported escape mutants that arose through a point mutation, have a point mutation at this position (Das et al., 2003; Boden et al., 2003; and Gitlin et al., 2002). Large-scale testing of all the effective shRNAs we have found confirms this preference and/or shows that mutations at other positions result in effective evasion from RNAi. Combining the primary shRNA with a second generation anticipating this escape option(s) prevents escape.

Also, we have recently identified a novel resistance mechanism through the formation of stable RNA structure by (a) mutation(s) in or near the targeted sequence. This knowledge, combined with computer-assisted RNA structure prediction, leads to a better design of a set of second generation shRNAs.

### Example 3

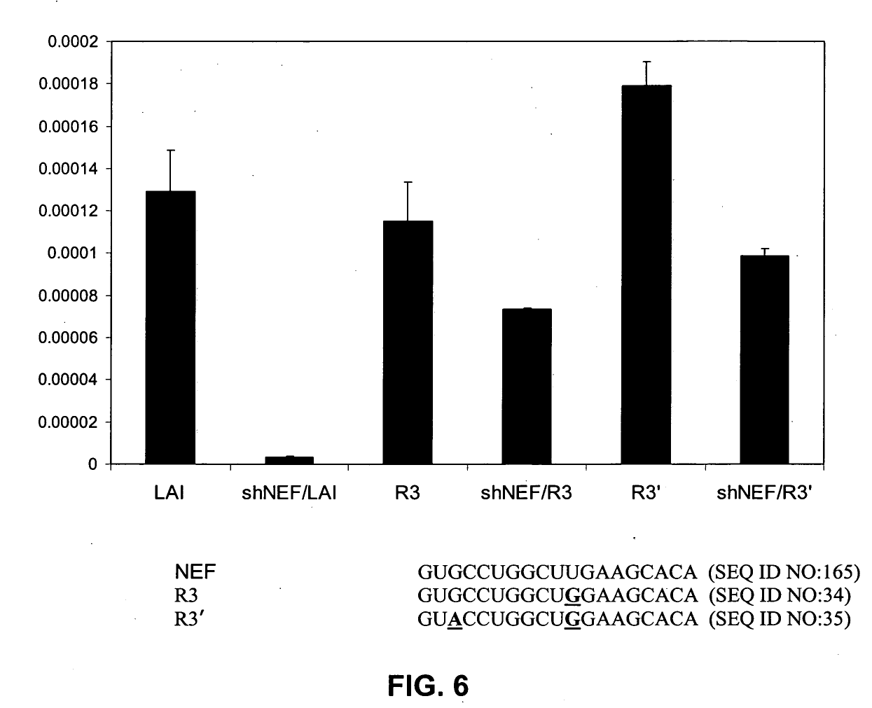

To test if the principle of anticipating escape is feasible, we tested this concept for two escape mutants against shNEF (Das et al., 2003). We designed complementary shRNAs targeting the escape mutants R3 and R3′. In a co-transfection experiment, LAI was strongly inhibited by the shNEF, while the molecular clones of the escape mutants R3 and R3′ were almost insensitive (FIG. 6). The inhibition was about two-fold, which is executed at the level of translational silencing or at the level of RNAi. When we co-transfected these escape mutants with their complementary shRNAs, inhibition was restored to normal levels (FIG. 7). Therefore, as a proof of concept, we have shown that it is possible to counteract escape by designing a second generation shRNA complementary to an escape mutant.

### Example 4

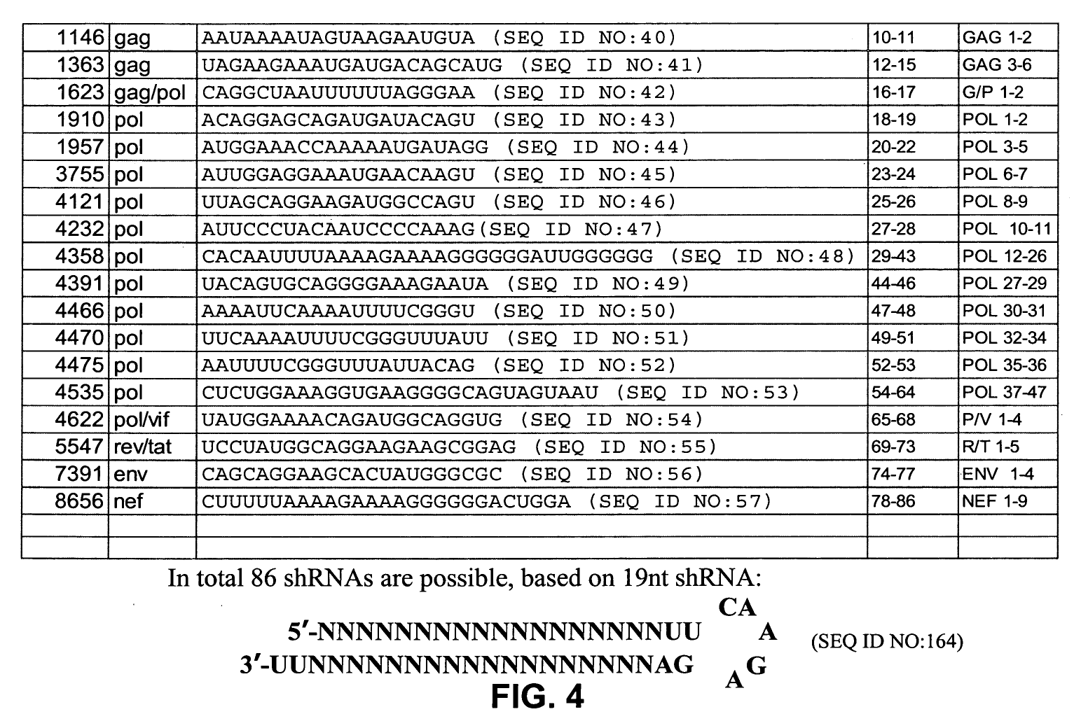

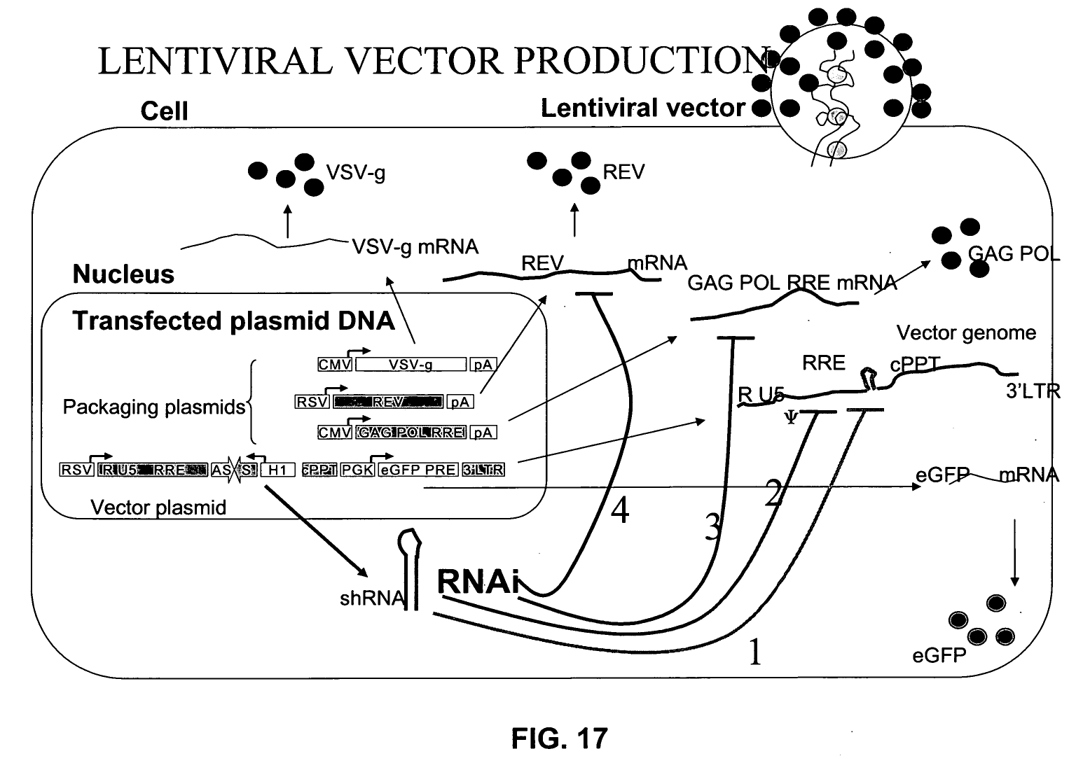

A preferred vector delivery system is the lentiviral vector system, which is actually based on HIV-1 itself (Zufferey et al., 1997). This vector system is particularly suited to stably transduce hematopoietic stem cells or CD4+ T cells, which are prime candidates for a gene therapy approach to treat HIV-1 infection. This vector system obviously contains HIV-1 sequences. These may also contain the conserved sequences presented in FIG. 4 and Table 1A. When RNA molecules (antisense, ribozyme, siRNA, etc.) acting against these HIV-1 sequences are expressed from the lentiviral vector, it can be expected that production of such a viral vector is severely reduced (FIG. 17). This effect will become even more severe when multiple RNA molecules are expressed from a single vector.

In order to restore vector production, target sequences present within the vector production system need to be altered such that sequence-specific inhibitory effects are released while not interfering with the viral vector system. For example, for lentiviral vector production, GagPol gene expression is required. Conventional production involves a GagPol construct expressing the original HIV-1 sequence. An alternative production plasmid involves the use of a GagPol expression plasmid that has been optimized for human codon usage, which is thus quite different from the HIV-1 sequence (Kotsopoulou et al., 2000; Wagner et al., 2000). It has been shown that lentiviral vector production with such a construct is similar to production with the conventional plasmid. Therefore, such a plasmid is ideally suited for lentiviral vector production involving multiple shRNAs (or other molecules) against highly conserved GagPol sequences.

Another problem is associated with the presence of any shRNA expression cassette in a lentiviral vector. The target is always present within the sequence that encodes for the shRNA (an antisense RNAi strand that is base-paired to the target sense strand in the hairpin), thus is also part of the vector genome (FIG. 17, route 1). Nobody to date has reported any problem with lentiviral vectors containing expression cassettes for shRNAs, indicating that this “self-targeting” is not a problem. Recently, it was shown that when a target sequence is contained within a hairpin structure, it is no longer a target for RNAi (Westerhout et al., 2005). This explains why reduced titers are in general never observed, since the shRNA encoding sequence forms a very stable RNA hairpin structure.

### Example 5

### Materials and Methods

### Plasmid Construction

Lentiviral vector plasmids are derived from the construct pRRLcpptpgkgfppreSsin (Seppen et al., 2002), which we renamed JS1. Expression cassettes for shRNAs were obtained by digestion of pSUPER constructs with XhoI and PstI and inserted into the XhoI and PstI site of JS1, resulting in plasmids JS1-shRNA. Multiple shRNA-expressing lentiviral vectors were constructed as follows. First, the expression cassette insert for shPol-47 was obtained by digestion of pSUPER shPol-47 with SmaI and XhoI and inserted in between the HindIII and XhoI sites of the pSUPER shGag-5 plasmid, resulting in pSUPER-shRNA2. A third cassette for shPol-1 was inserted by repeating this procedure, thereby obtaining pSUPER-shRNA3. The double (shPol-47 and shGag-5) and triple (shPol-1, shPol-47 and shGag-5) expression cassettes were digested with SmaI and XhoI and inserted into the XhoI and EcoRV sites of JS-1, resulting in the JS1-shRNA2 and JS1-shRNA3 constructs, respectively.

Firefly reporter plasmids were constructed by insertion of an annealed target HIV-1 target sequence of 50 to 70 nucleotides, with the 19 nucleotide target region placed in the middle, in the EcoRI and PstI sites of pGL3-Nef (Westerhout et al., 2005).

### Cell Culture

Human embryonic kidney (HEK) 203T adherent cells were grown in DMEM (Gibco BRL) and SupT1 suspension cells were grown in RPMI (Gibco BRL), both supplemented with 10% fetal calf serum, penicillin (100 U/m) and streptomycin (100 μg/ml) in a humidified chamber at 37° C. and 5% CO₂.

### Lentiviral Vector Production

Lentiviral vector production was performed as follows. 2.2×10⁶ 293T cells were seeded in a T25 flask the day prior to transfection. The next day, medium was replaced with 2.2 ml medium without antibiotics. Subsequently, lentiviral vector plasmid (2.4 μg) was co-transfected with packaging plasmids pSYNGP (1.5 μg) (Kotsopoulou et al., 2000), RSV-rev (0.6 μg) and pVSVg (0.8 μg) (Dull et al., 1998; Zufferey et al., 1998) using 16 μl lipofectamine-2000 and 1.5 ml Optimem (Gibco BRL). The second day, medium was replaced with fresh medium. On the third and fourth days, medium containing lentiviral vector was harvested in the morning. Cellular debris was removed by low-speed centrifugation and supernatant was stored at 4° C. On the fourth day, supernatants were pooled and aliquots of 0.8 ml were stored at −80° C.

### Cell Transduction

Cells were transduced at low multiplicity of infection, which means that the percentage of transduction was always kept below 30%, preferably at 10%. After transduction, GFP-positive cells were selected with life FACS sorting. Selected polyclonal 293T cell or SupT1 cells were transfected with pLAI or infected with LAI virus, respectively.

Transfection of pLUC-Reporters or pLAI in Stable 293T Cell Lines

Smaller scale transfections were carried out in a 24-well format. Per well, 1-1.5×10⁵ 293T cells were seeded in 500 ml DMEM with 10% FCS without antibiotics. The next day, 100 ng of pLAI or pLUC-reporter and 2.5 ng of pRL (Renilla Luciferase) was transfected with 1 to 2 μl lipofectamine-2000 in a reaction volume of 100 μl according to the manufacturer's instructions. Eight hours post-transfection, the medium was replaced with 200 μl medium containing antibiotics. Forty-eight hours after transfection, medium samples were taken for CA-p24 ELISA if pLAI was transfected and cells were lysed for Renilla measurements and Luciferase measurements if pLUC-reporters were transfected. CA-p24 is a measure of virus production. To correct for variations in transfections, relative CA-24 values were determined by dividing the CA-p24 measurements by the Renilla values obtained.

### HIV-1 LAI Infections

Virus was produced by transfection of the molecular clone pLAI in 293T cells. In a 24-well plate, 1 ml of SupT1 cells or selected cell lines were infected with an equal amount of virus, which corresponded to 1 ng of CA-p24. Every two days, CA-2p4 was measured in the cellular supernatant.

### Results

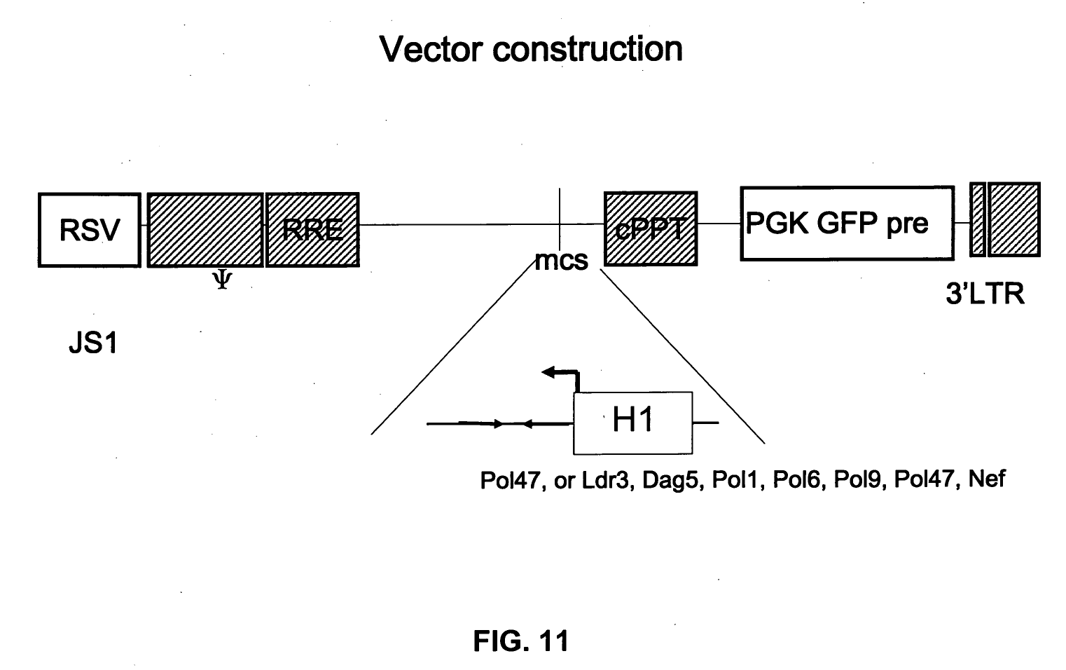

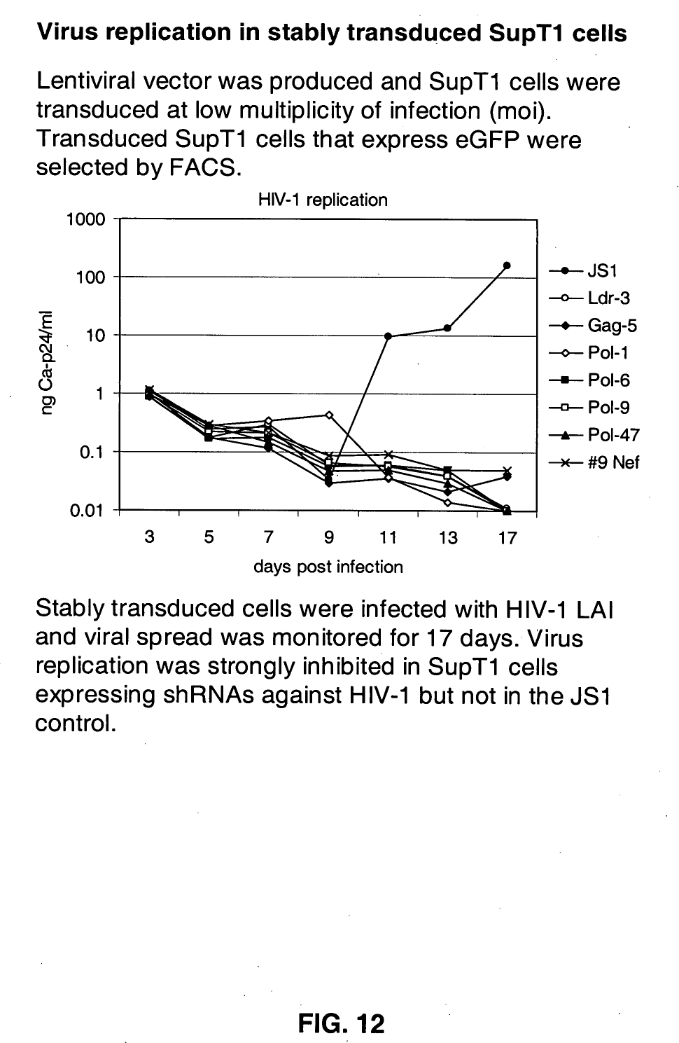

A selection of some of the strongly inhibiting shRNAs (Table 2A) was shown to efficiently inhibit HIV-1 in replication setting. The SupT1 T cell line was transduced at a low multiplicity of infection with lentiviral vectors containing a single shRNA expression cassette (FIG. 11). We tested seven shRNAs and included the shRNA against HIV-1 Nef as a positive control and the empty vector JS1 as a negative control. The lentiviral vector contains an expression cassette for GFP (Seppen et al., 2002), and transduced cells were selected as GFP-positive cells with life FACS sorting. All shRNA-transduced cells were strongly inhibiting HIV-1 replication (FIG. 12). The JS1 control cells showed normal HIV-1 replication as judged by massive syncytia formation and CA-p24 production (FIG. 12). These results confirm that the inhibition observed in the transient screen translates to effective inhibition of HIV-1 replication in a setting that is more relevant to the gene therapy goal.

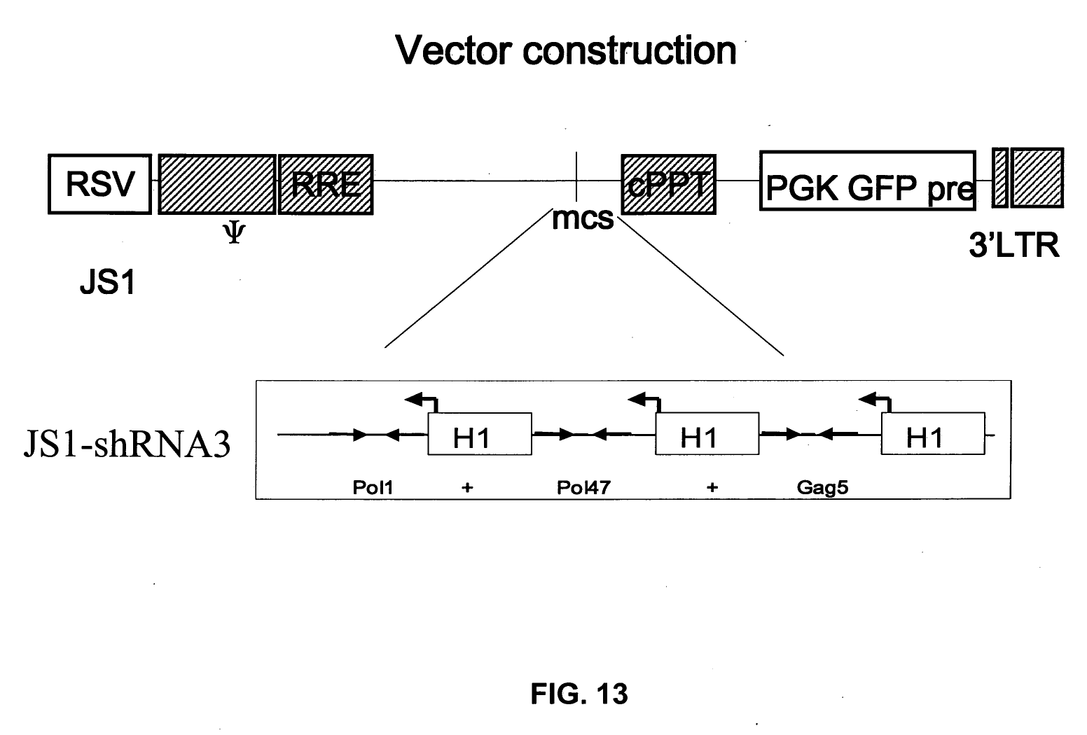

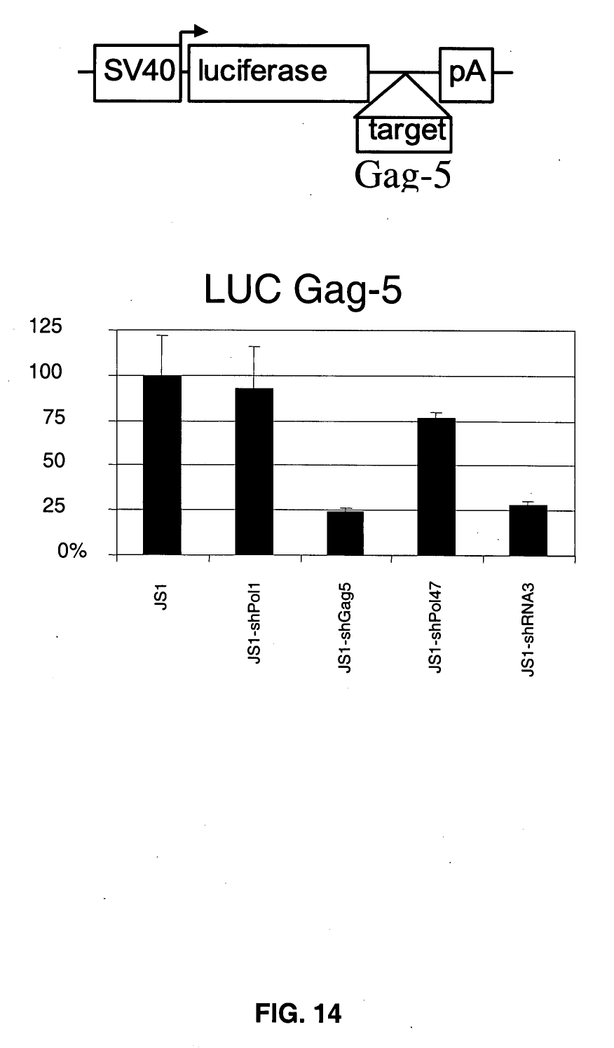

Towards a multi-shRNA gene therapy, we combined several shRNA expression cassettes in a single lentiviral vector construct. First, we inserted expression cassettes for shPol-1 and shPol-47 in pSUPER-shGag-5, resulting in a triple shRNA-expressing construct. Next, we cloned the triple shRNA expression cassettes in the lentiviral vector JS1, resulting in JS1-shRNA3 (FIG. 13). In initial transfection experiments with reporter constructs, we confirmed sequence-specific inhibition for each shRNA present in JS1-shRNA3. For instance, when pLuc-Gag-5 was co-transfected with different lentiviral vector constructs, only when shGag-5 was expressed, were Luciferase levels reduced (FIG. 14). More importantly, we observed that the individual activity of each shRNA was maintained when inhibition expressed from JS1-shRNA3 (FIG. 14 for Gag-5, results not shown for Pol-1 and Pol-47).

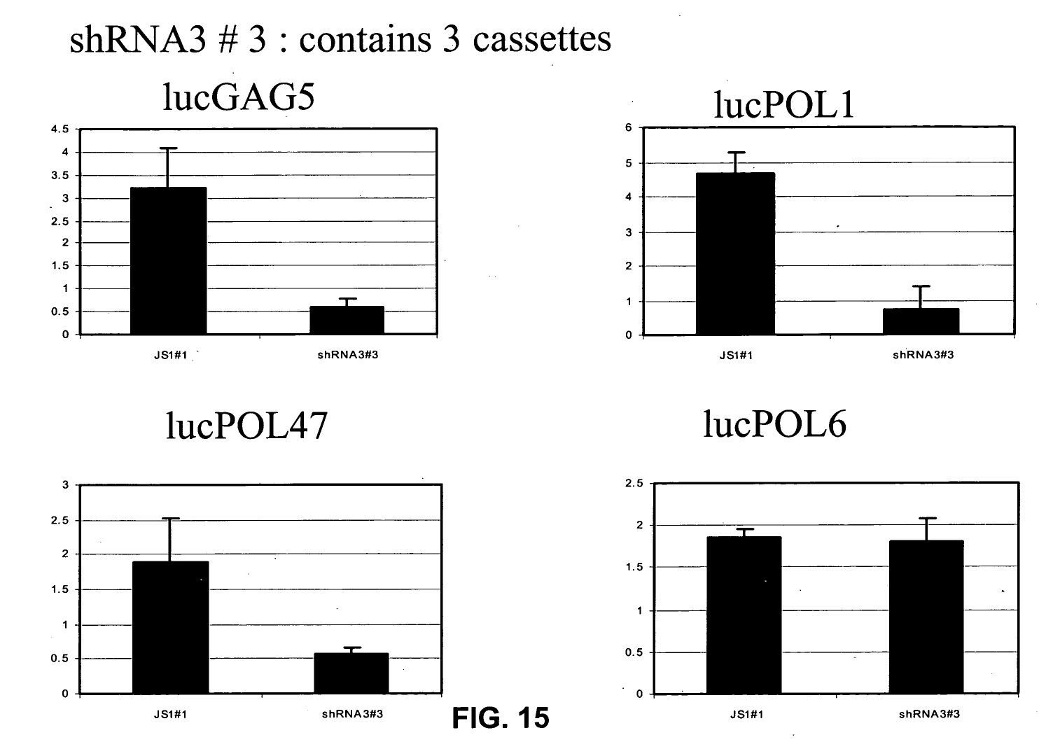

We next made stable cell lines with the triple-shRNA lentiviral vector. Cellular clones were selected that contain the triple shRNA cassette. We co-transfected Luciferase reporter constructs for Gag-5, Pol-1, Pol-47 and, as a control, Pol-6 in these cell clones and compared their expression with cell clones transduced with an empty vector. Transfection in cell clone #3, showed inhibition of all three reporters when compared to control cells transduced with the empty JS1 vector (FIG. 15). The control reporter Pol-6 was not affected (FIG. 15).

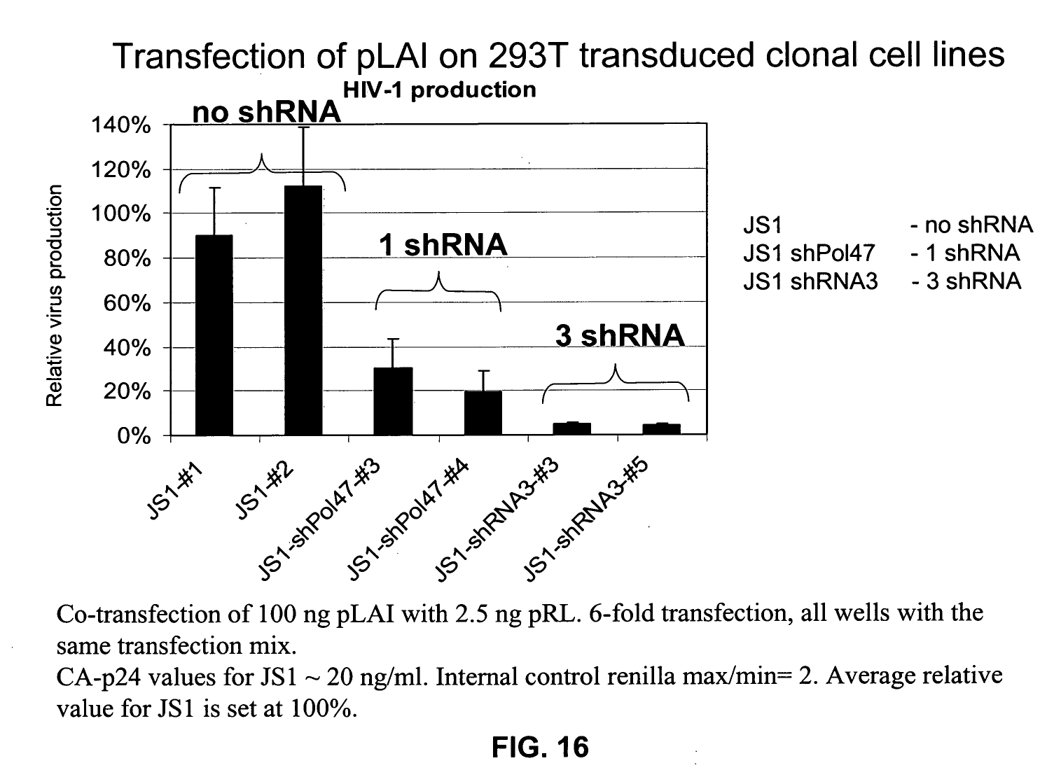

Finally, we tested HIV-1 virus production in the different clonal cell lines. We transfected pLAI in two clones, each with no, one or three shRNA expression cassettes. Clones with three different shRNA cassettes inhibited much stronger, about 20-fold, compared to clones with a single shRNA cassettes, which only inhibited four-fold (FIG. 16). Combined, our data show that the expression of three different shRNAs from a single vector is preferred and does not negatively influence the activity of each individual shRNA when compared to single shRNA vectors. The combination of multiple shRNAs results in an additive inhibition of HIV-1 production.

### REFERENCES

Anderson et al., Oligonucleotides 13:303-312 (2003).
Boden D., O. Pusch, F. Lee, L. Tucker, B. Ramratnam. J. Virol. 2003 November; 77(21):11531-5.
Das et al. Journal of Virology, Vol. 78, No. 5 (2004) pages 2601-2605.
Dull T., R. Zufferey, M. Kelly, R. J. Mandel, M. Nguyen, D. Trono, and L. Naldini. (1998). A third-generation lentivirus vector with a conditional packaging system. J. Virol. 72, 8463-8471.
Gitlin et al. Nature, Vol. 418 (2002) pages 430-434.
Kotsopoulou E., V. N. Kim, A. J. Kingsman, S. M. Kingsman, and K. A. Mitrophanous (2000). A Rev-independent human immunodeficiency virus type 1 (HIV-1)-based vector that exploits a codon-optimized HIV-1 gag-pol gene. J. Virol. 74, 4839-4852.
Seppen J., M. Rijnberg, M. P. Cooreman, and R. P. Oude Elferink (2002). Lentiviral vectors for efficient transduction of isolated primary quiescent hepatocytes. J. Hepatol. 36, 459-465.
Wagner R., M. Graf, K. Bieler, H. Wolf, T. Grunwald, P. Foley, and K. Uberla (2000). Rev-independent expression of synthetic gag-pol genes of human immunodeficiency virus type 1 and simian immunodeficiency virus: implications for the safety of lentiviral vectors. Hum. Gene Ther. 11, 2403-2413.
Westerhout E. M., M. Ooms, M. Vink, A. T. Das, and B. Berkhout (2005). HIV-1 can escape from RNA interference by evolving an alternative structure in its RNA genome. Nucleic Acids Res. 33, 796-804.
Zufferey R., T. Dull, R. J. Mandel, A. Bukovsky, D. Quiroz, L. Naldini and D. Trono (1998). Self-inactivating lentivirus vector for safe and efficient in vivo gene delivery. J. Virol. 72, 9873-9880.
Zufferey R., D. Nagy, R. J. Mandel, L. Naldini, and D. Trono (1997). Multiply attenuated lentiviral vector achieves efficient gene delivery in vivo. Nat. Biotechnol. 15, 871-875.

1. A nucleic acid molecule comprising a sequence that is complementary to a mutant of a genomic nucleic acid sequence of an organism capable of mutating its genome.

2. The nucleic acid molecule according to claim 1, wherein said organism comprises a virus.

3. The nucleic acid molecule of claim 2, wherein said organism comprises HIV, Hepatitis B, Hepatitis C, SARS, Ebola, Influenza, West Nile and/or polio virus.

4. The nucleic acid molecule of claim 1, wherein said genomic nucleic acid sequence of said organism comprises at least part of an essential region.

5. The nucleic acid molecule of claim 1, wherein said genomic nucleic acid sequence of said organism comprises at least part of a conserved region.

6. The nucleic acid molecule according to claim 5, wherein said conserved region comprises a region as depicted in Table 1B.

7. The nucleic acid molecule of claim 1, wherein said genomic nucleic acid sequence of said organism comprises at least one coding sequence and/or at least one DNA/RNA regulatory sequence.

8. The nucleic acid molecule of claim 1, wherein said mutant comprises a coding sequence of said organism wherein a nucleotide of a codon is substituted such that the resulting codon encodes the same amino acid residue.

9. The nucleic acid molecule of claim 1, wherein said mutant comprises a genomic nucleic acid sequence of said organism with at least one mutation resulting in a significantly altered structure of said genomic nucleic acid sequence.

10. The nucleic acid molecule of claim 1, comprising small interfering RNA.

11. The nucleic acid molecule of claim 1, comprising short hairpin RNA.

12. The nucleic acid molecule of claim 1, comprising an inducible repressor and/or activator.

13. The nucleic acid molecule of claim 1, comprising a sequence that is complementary to a mutant of a sequence as depicted in Tables 2A, 2B or 2C, or a functional part, derivative and/or analogue thereof.

14. The nucleic acid molecule of claim 1, comprising a sequence that is complementary to a mutant of a sequence as depicted in Tables 2A or 2B, or a functional part, derivative and/or analogue thereof.

15. The nucleic acid molecule of claim 1, comprising a sequence that is complementary to a mutant of a sequence as depicted in Table 2A, or a functional part, derivative and/or analogue thereof.

16. The nucleic acid molecule of claim 1, that is complementary to the sequence GUGCCUGGCUGGAAGCACA (SEQ ID NO:34) and/or GUACCUGGCUGGAAGCACA (SEQ ID NO:35), or a functional part, derivative and/or analogue thereof.

17. A nucleic acid molecule comprising a sequence that is complementary to a conserved region as depicted in Table 1B, or a functional part, derivative and/or analogue thereof.

18. The nucleic acid according to claim 17, comprising a sequence that is complementary to a sequence as depicted in Tables 2A or 2B, or a functional part, derivative and/or analogue thereof.

19. The nucleic acid of claim 17, comprising a sequence that is complementary to a sequence as depicted in Table 2A, or a functional part, derivative and/or analogue thereof.

20. The nucleic acid molecule of claim 1, which is complementary to the sequence GGGGGGACUGGAAGGGCUA (SEQ ID NO:154), GGGGGACUGGAAGGGCUAA (SEQ ID NO:155), GGGGACUGGAAGGGCUAAU (SEQ ID NO:156) and/or GGGACUGGAAGGGCUAAUU (SEQ ID NO:157).

21. (canceled)

22. (canceled)

23. (canceled)

24. A pharmaceutical composition comprising:
the nucleic acid molecule of claim 20, and a suitable diluent or carrier.

25. The pharmaceutical composition of claim 24, further comprising a nucleic acid molecule comprising a sequence that is complementary to said genomic nucleic acid sequence of said organism.

26. The pharmaceutical composition of claim 24, further comprising another kind of drug.

27. The pharmaceutical composition of claim 24, comprising nucleic acid sequences that are complementary to mutants of at least two genomic nucleic acid sequences of said organism.

28. The pharmaceutical composition of claim 24, comprising nucleic acid sequences that are complementary to mutants of at least six genomic nucleic acid sequences of said organism.

29. A gene delivery vehicle comprising the nucleic acid sequence of claim 1.

30. A method for at least in part counteracting an organism, comprising providing said organism with the nucleic acid molecule of claim 1.

31. The method according to claim 30, wherein said organism is additionally provided with a nucleic acid molecule comprising a sequence that is complementary to said genomic nucleic acid sequence of said organism.

32. The method according to claim 30, wherein said organism is provided with nucleic acid sequences that are complementary to mutants of at least two genomic nucleic acid sequences of said organism.

33. The method according to claim 30, wherein said organism is provided with nucleic acid sequences that are complementary to mutants of at least six genomic nucleic acid sequences of said organism.

34. A method for interfering with a cell comprising:
an organism capable of mutating its genome and/or
a nucleic acid sequence of an organism capable of mutating its genome, said method comprising:
providing said cell with the nucleic acid sequence of claim 1.

35. A method for at least in part treating or preventing a disease involved with an organism capable of mutating its genome, said method comprising:
providing an individual suffering from, or at risk of suffering from, said disease with the gene delivery vehicle according to claim 29.

36. The method according to claim 34, further comprising providing said cell with another kind of drug capable of at least in part counteracting said organism.

37. A kit of parts comprising the nucleic acid molecule of claim 1, and another kind of drug capable of at least in part counteracting said organism.


SEQUENCE LISTING (171 sequences)

SEQ ID NO: 1 - DNA, 19 bp, Human immunodeficiency virus
aggagagagatgggtgcga

SEQ ID NO: 2 - DNA, 19 bp, Human immunodeficiency virus
ggagagagatgggtgcgag

SEQ ID NO: 3 - DNA, 19 bp, Human immunodeficiency virus
gagagagatgggtgcgaga

SEQ ID NO: 4 - DNA, 19 bp, Human immunodeficiency virus
agagagatgggtgcgagag

SEQ ID NO: 5 - DNA, 19 bp, Human immunodeficiency virus
gagagatgggtgcgagagc

SEQ ID NO: 6 - DNA, 19 bp, Human immunodeficiency virus
agagatgggtgcgagagcg

SEQ ID NO: 7 - DNA, 19 bp, Human immunodeficiency virus
gagatgggtgcgagagcgt

SEQ ID NO: 8 - DNA, 19 bp, Human immunodeficiency virus
agatgggtgcgagagcgtc

SEQ ID NO: 9 - DNA, 19 bp, Human immunodeficiency virus
agaagaaatgatgacagca

SEQ ID NO: 10 - DNA, 19 bp, Human immunodeficiency virus
gaagaaatgatgacagcat

SEQ ID NO: 11 - DNA, 19 bp, Human immunodeficiency virus
aagaaatgatgacagcatg

SEQ ID NO: 12 - DNA, 19 bp, Human immunodeficiency virus
acaggagcagatgatacag

SEQ ID NO: 13 - DNA, 19 bp, Human immunodeficiency virus
caggagcagatgatacagt

SEQ ID NO: 14 - DNA, 19 bp, Human immunodeficiency virus
attggaggaaatgaacaag

SEQ ID NO: 15 - DNA, 19 bp, Human immunodeficiency virus
tagcaggaagatggccagt

SEQ ID NO: 16 - DNA, 19 bp, Human immunodeficiency virus
cagtgcaggggaaagaata

SEQ ID NO: 17 - DNA, 19 bp, Human immunodeficiency virus
aggtgaaggggcagtagta

SEQ ID NO: 18 - DNA, 19 bp, Human immunodeficiency virus
gtgaaggggcagtagtaat

SEQ ID NO: 19 - RNA, 19 nt, Human immunodeficiency virus
ggaaaacagauggcaggug

SEQ ID NO: 20 - DNA, 19 bp, Human immunodeficiency virus
tatggcaggaagaagcgga

SEQ ID NO: 21 - DNA, 19 bp, Human immunodeficiency virus
atggcaggaagaagcggag

SEQ ID NO: 22 - DNA, 19 bp, Human immunodeficiency virus
aaggagagagatgggtgcg

SEQ ID NO: 23 - DNA, 19 bp, Human immunodeficiency virus
tagaagaaatgatgacagc

SEQ ID NO: 24 - DNA, 19 bp, Human immunodeficiency virus
ttagcaggaagatggccag

SEQ ID NO: 25 - DNA, 19 bp, Human immunodeficiency virus
tagcaggaagatggccagt

SEQ ID NO: 26 - DNA, 19 bp, Human immunodeficiency virus
attccctacaatccccaaa

SEQ ID NO: 27 - DNA, 19 bp, Human immunodeficiency virus
acaattttaaaagaaaagg

SEQ ID NO: 28 - DNA, 19 bp, Human immunodeficiency virus
tacagtgcaggggaaagaa

SEQ ID NO: 29 - DNA, 19 bp, Human immunodeficiency virus
aaaattcaaaattttcggg

SEQ ID NO: 30 - DNA, 19 bp, Human immunodeficiency virus
ggaaaggtgaaggggcagt

SEQ ID NO: 31 - DNA, 19 bp, Human immunodeficiency virus
gaaaggtgaaggggcagta

SEQ ID NO: 32 - DNA, 19 bp, Human immunodeficiency virus
aaggtgaaggggcagtagt

SEQ ID NO: 33 - DNA, 19 bp, Human immunodeficiency virus
ctttttaaaagaaaagggg

SEQ ID NO: 34 - RNA, 19 nt, Artificial
  sequence capable of inhibiting HIV escape mutant
gugccuggcuggaagcaca

SEQ ID NO: 35 - RNA, 19 nt, Artificial
  sequence capable of inhibiting HIV escape mutant
guaccuggcuggaagcaca

SEQ ID NO: 36 - DNA, 64 bp, Artificial
  oligo sequence
gatccccnnnnnnnnnnnnnnnnnnnttcaagagannnnnnnnnnnnnnnnnnntttttggaaa

SEQ ID NO: 37 - DNA, 64 bp, Artificial
  oligo sequence
agcttttccaaaaannnnnnnnnnnnnnnnnnntctcttgaannnnnnnnnnnnnnnnnnnggg

SEQ ID NO: 38 - RNA, 27 nt, Human immunodeficiency virus
gaaaaggggggacuggaagggcuaauu

SEQ ID NO: 39 - RNA, 27 nt, Human immunodeficiency virus
aaggagagagaugggugcgagagcguc

SEQ ID NO: 40 - RNA, 20 nt, Human immunodeficiency virus
aauaaaauaguaagaaugua

SEQ ID NO: 41 - RNA, 22 nt, Human immunodeficiency virus
uagaagaaaugaugacagcaug

SEQ ID NO: 42 - RNA, 20 nt, Human immunodeficiency virus
caggcuaauuuuuuagggaa

SEQ ID NO: 43 - RNA, 20 nt, Human immunodeficiency virus
acaggagcagaugauacagu

SEQ ID NO: 44 - RNA, 21 nt, Human immunodeficiency virus
auggaaaccaaaaaugauagg

SEQ ID NO: 45 - RNA, 20 nt, Human immunodeficiency virus
auuggaggaaaugaacaagu

SEQ ID NO: 46 - RNA, 20 nt, Human immunodeficiency virus
uuagcaggaagauggccagu

SEQ ID NO: 47 - RNA, 20 nt, Human immunodeficiency virus
auucccuacaauccccaaag

SEQ ID NO: 48 - RNA, 33 nt, Human immunodeficiency virus
cacaauuuuaaaagaaaaggggggauugggggg

SEQ ID NO: 49 - RNA, 21 nt, Human immunodeficiency virus
uacagugcaggggaaagaaua

SEQ ID NO: 50 - RNA, 20 nt, Human immunodeficiency virus
aaaauucaaaauuuucgggu

SEQ ID NO: 51 - RNA, 21 nt, Human immunodeficiency virus
uucaaaauuuucggguuuauu

SEQ ID NO: 52 - RNA, 20 nt, Human immunodeficiency virus
aauuuucggguuuauuacag

SEQ ID NO: 53 - RNA, 29 nt, Human immunodeficiency virus
cucuggaaaggugaaggggcaguaguaau

SEQ ID NO: 54 - RNA, 22 nt, Human immunodeficiency virus
uauggaaaacagauggcaggug

SEQ ID NO: 55 - RNA, 23 nt, Human immunodeficiency virus
uccuauggcaggaagaagcggag

SEQ ID NO: 56 - RNA, 22 nt, Human immunodeficiency virus
cagcaggaagcacuaugggcgc

SEQ ID NO: 57 - RNA, 27 nt, Human immunodeficiency virus
cuuuuuaaaagaaaaggggggacugga

SEQ ID NO: 58 - RNA, 25 nt, Human immunodeficiency virus
aggagagagaugggugcgagagcgu

SEQ ID NO: 59 - RNA, 21 nt, Human immunodeficiency virus
uagaagaaaugaugacagcau

SEQ ID NO: 60 - RNA, 19 nt, Human immunodeficiency virus
auuggaggaaaugaacaag

SEQ ID NO: 61 - RNA, 19 nt, Human immunodeficiency virus
uagcaggaagauggccagu

SEQ ID NO: 62 - RNA, 19 nt, Human immunodeficiency virus
uacagugcaggggaaagaa

SEQ ID NO: 63 - RNA, 19 nt, Human immunodeficiency virus
cagugcaggggaaagaaua

SEQ ID NO: 64 - RNA, 20 nt, Human immunodeficiency virus
aaggugaaggggcaguagua

SEQ ID NO: 65 - RNA, 19 nt, Human immunodeficiency virus
gugaaggggcaguaguaau

SEQ ID NO: 66 - RNA, 19 nt, Human immunodeficiency virus
ggaaaacagauggcaggug

SEQ ID NO: 67 - RNA, 20 nt, Human immunodeficiency virus
uauggcaggaagaagcggag

SEQ ID NO: 68 - RNA, 19 nt, Human immunodeficiency virus
aggagagagaugggugcga

SEQ ID NO: 69 - RNA, 19 nt, Human immunodeficiency virus
ggagagagaugggugcgag

SEQ ID NO: 70 - RNA, 19 nt, Human immunodeficiency virus
gagagagaugggugcgaga

SEQ ID NO: 71 - RNA, 19 nt, Human immunodeficiency virus
agagagaugggugcgagag

SEQ ID NO: 72 - RNA, 19 nt, Human immunodeficiency virus
gagagaugggugcgagagc

SEQ ID NO: 73 - RNA, 19 nt, Human immunodeficiency virus
agagaugggugcgagagcg

SEQ ID NO: 74 - RNA, 19 nt, Human immunodeficiency virus
gagaugggugcgagagcgu

SEQ ID NO: 75 - RNA, 19 nt, Human immunodeficiency virus
agaugggugcgagagcguc

SEQ ID NO: 76 - RNA, 19 nt, Human immunodeficiency virus
agaagaaaugaugacagca

SEQ ID NO: 77 - RNA, 19 nt, Human immunodeficiency virus
gaagaaaugaugacagcau

SEQ ID NO: 78 - RNA, 19 nt, Human immunodeficiency virus
aagaaaugaugacagcaug

SEQ ID NO: 79 - RNA, 19 nt, Human immunodeficiency virus
acaggagcagaugauacag

SEQ ID NO: 80 - RNA, 19 nt, Human immunodeficiency virus
caggagcagaugauacagu

SEQ ID NO: 81 - RNA, 19 nt, Human immunodeficiency virus
aggugaaggggcaguagua

SEQ ID NO: 82 - RNA, 19 nt, Human immunodeficiency virus
uauggcaggaagaagcgga

SEQ ID NO: 83 - RNA, 19 nt, Human immunodeficiency virus
auggcaggaagaagcggag

SEQ ID NO: 84 - RNA, 19 nt, Human immunodeficiency virus
aaggagagagaugggugcg

SEQ ID NO: 85 - RNA, 19 nt, Human immunodeficiency virus
uagaagaaaugaugacagc

SEQ ID NO: 86 - RNA, 19 nt, Human immunodeficiency virus
uuagcaggaagauggccag

SEQ ID NO: 87 - RNA, 19 nt, Human immunodeficiency virus
auucccuacaauccccaaa

SEQ ID NO: 88 - RNA, 19 nt, Human immunodeficiency virus
acaauuuuaaaagaaaagg

SEQ ID NO: 89 - RNA, 19 nt, Human immunodeficiency virus
uacagugcaggggaaagaa

SEQ ID NO: 90 - RNA, 19 nt, Human immunodeficiency virus
aaaauucaaaauuuucggg

SEQ ID NO: 91 - RNA, 19 nt, Human immunodeficiency virus
ggaaaggugaaggggcagu

SEQ ID NO: 92 - RNA, 19 nt, Human immunodeficiency virus
gaaaggugaaggggcagua

SEQ ID NO: 93 - RNA, 19 nt, Human immunodeficiency virus
aaggugaaggggcaguagu

SEQ ID NO: 94 - RNA, 19 nt, Human immunodeficiency virus
cuuuuuaaaagaaaagggg

SEQ ID NO: 95 - RNA, 19 nt, Human immunodeficiency virus
aauaaaauaguaagaaugu

SEQ ID NO: 96 - RNA, 19 nt, Human immunodeficiency virus
auaaaauaguaagaaugua

SEQ ID NO: 97 - RNA, 19 nt, Human immunodeficiency virus
caggcuaauuuuuuaggga

SEQ ID NO: 98 - RNA, 19 nt, Human immunodeficiency virus
aggcuaauuuuuuagggaa

SEQ ID NO: 99 - RNA, 19 nt, Human immunodeficiency virus
auggaaaccaaaaaugaua

SEQ ID NO: 100 - RNA, 19 nt, Human immunodeficiency virus
uggaaaccaaaaaugauag

SEQ ID NO: 101 - RNA, 19 nt, Human immunodeficiency virus
ggaaaccaaaaaugauagg

SEQ ID NO: 102 - RNA, 19 nt, Human immunodeficiency virus
uuggaggaaaugaacaagu

SEQ ID NO: 103 - RNA, 19 nt, Human immunodeficiency virus
uucccuacaauccccaaag

SEQ ID NO: 104 - RNA, 19 nt, Human immunodeficiency virus
cacaauuuuaaaagaaaag

SEQ ID NO: 105 - RNA, 19 nt, Human immunodeficiency virus
caauuuuaaaagaaaaggg

SEQ ID NO: 106 - RNA, 19 nt, Human immunodeficiency virus
aauuuuaaaagaaaagggg

SEQ ID NO: 107 - RNA, 19 nt, Human immunodeficiency virus
auuuuaaaagaaaaggggg

SEQ ID NO: 108 - RNA, 19 nt, Human immunodeficiency virus
uuuuaaaagaaaagggggg

SEQ ID NO: 109 - RNA, 19 nt, Human immunodeficiency virus
uuuaaaagaaaagggggga

SEQ ID NO: 110 - RNA, 19 nt, Human immunodeficiency virus
uuaaaagaaaaggggggau

SEQ ID NO: 111 - RNA, 19 nt, Human immunodeficiency virus
uaaaagaaaaggggggauu

SEQ ID NO: 112 - RNA, 19 nt, Human immunodeficiency virus
aaaagaaaaggggggauug

SEQ ID NO: 113 - RNA, 19 nt, Human immunodeficiency virus
aaagaaaaggggggauugg

SEQ ID NO: 114 - RNA, 19 nt, Human immunodeficiency virus
aagaaaaggggggauuggg

SEQ ID NO: 115 - RNA, 19 nt, Human immunodeficiency virus
agaaaaggggggauugggg

SEQ ID NO: 116 - RNA, 19 nt, Human immunodeficiency virus
gaaaaggggggauuggggg

SEQ ID NO: 117 - RNA, 19 nt, Human immunodeficiency virus
aaaaggggggauugggggg

SEQ ID NO: 118 - RNA, 19 nt, Human immunodeficiency virus
acagugcaggggaaagaau

SEQ ID NO: 119 - RNA, 19 nt, Human immunodeficiency virus
aaauucaaaauuuucgggu

SEQ ID NO: 120 - RNA, 19 nt, Human immunodeficiency virus
uucaaaauuuucggguuua

SEQ ID NO: 121 - RNA, 19 nt, Human immunodeficiency virus
ucaaaauuuucggguuuau

SEQ ID NO: 122 - RNA, 19 nt, Human immunodeficiency virus
caaaauuuucggguuuauu

SEQ ID NO: 123 - RNA, 19 nt, Human immunodeficiency virus
aauuuucggguuuauuaca

SEQ ID NO: 124 - RNA, 19 nt, Human immunodeficiency virus
auuuucggguuuauuacag

SEQ ID NO: 125 - RNA, 19 nt, Human immunodeficiency virus
cucuggaaaggugaagggg

SEQ ID NO: 126 - RNA, 19 nt, Human immunodeficiency virus
ucuggaaaggugaaggggc

SEQ ID NO: 127 - RNA, 19 nt, Human immunodeficiency virus
cuggaaaggugaaggggca

SEQ ID NO: 128 - RNA, 19 nt, Human immunodeficiency virus
uggaaaggugaaggggcag

SEQ ID NO: 129 - RNA, 19 nt, Human immunodeficiency virus
aaaggugaaggggcaguag

SEQ ID NO: 130 - RNA, 19 nt, Human immunodeficiency virus
ggugaaggggcaguaguaa

SEQ ID NO: 131 - RNA, 19 nt, Human immunodeficiency virus
uauggaaaacagauggcag

SEQ ID NO: 132 - RNA, 19 nt, Human immunodeficiency virus
auggaaaacagauggcagg

SEQ ID NO: 133 - RNA, 19 nt, Human immunodeficiency virus
uggaaaacagauggcaggu

SEQ ID NO: 134 - RNA, 19 nt, Human immunodeficiency virus
uccuauggcaggaagaagc

SEQ ID NO: 135 - RNA, 19 nt, Human immunodeficiency virus
ccuauggcaggaagaagcg

SEQ ID NO: 136 - RNA, 19 nt, Human immunodeficiency virus
cuauggcaggaagaagcgg

SEQ ID NO: 137 - RNA, 19 nt, Human immunodeficiency virus
cagcaggaagcacuauggg

SEQ ID NO: 138 - RNA, 19 nt, Human immunodeficiency virus
agcaggaagcacuaugggc

SEQ ID NO: 139 - RNA, 19 nt, Human immunodeficiency virus
gcaggaagcacuaugggcg

SEQ ID NO: 140 - RNA, 19 nt, Human immunodeficiency virus
caggaagcacuaugggcgc

SEQ ID NO: 141 - RNA, 19 nt, Human immunodeficiency virus
uuuuuaaaagaaaaggggg

SEQ ID NO: 142 - RNA, 19 nt, Human immunodeficiency virus
uuuuaaaagaaaagggggg

SEQ ID NO: 143 - RNA, 19 nt, Human immunodeficiency virus
uuuaaaagaaaagggggga

SEQ ID NO: 144 - RNA, 19 nt, Human immunodeficiency virus
uuaaaagaaaaggggggac

SEQ ID NO: 145 - RNA, 19 nt, Human immunodeficiency virus
uaaaagaaaaggggggacu

SEQ ID NO: 146 - RNA, 19 nt, Human immunodeficiency virus
aaaagaaaaggggggacug

SEQ ID NO: 147 - RNA, 19 nt, Human immunodeficiency virus
aaagaaaaggggggacugg

SEQ ID NO: 148 - RNA, 19 nt, Human immunodeficiency virus
aagaaaaggggggacugga

SEQ ID NO: 149 - RNA, 19 nt, Human immunodeficiency virus
gaaaaggggggacuggaag

SEQ ID NO: 150 - RNA, 19 nt, Human immunodeficiency virus
aaaaggggggacuggaagg

SEQ ID NO: 151 - RNA, 19 nt, Human immunodeficiency virus
aaaggggggacuggaaggg

SEQ ID NO: 152 - RNA, 19 nt, Human immunodeficiency virus
aaggggggacuggaagggc

SEQ ID NO: 153 - RNA, 19 nt, Human immunodeficiency virus
aggggggacuggaagggcu

SEQ ID NO: 154 - RNA, 19 nt, Human immunodeficiency virus
ggggggacuggaagggcua

SEQ ID NO: 155 - RNA, 19 nt, Human immunodeficiency virus
gggggacuggaagggcuaa

SEQ ID NO: 156 - RNA, 19 nt, Human immunodeficiency virus
ggggacuggaagggcuaau

SEQ ID NO: 157 - RNA, 19 nt, Human immunodeficiency virus
gggacuggaagggcuaauu

SEQ ID NO: 158 - RNA, 49 nt, Artificial
  sh RNA
gugccuggcuagaagcacauucaagagaugugcuucuagccaggcacuu

SEQ ID NO: 159 - RNA, 21 nt, Artificial
  sense strand of siRNA
gugccuggcuagaagcacauu

SEQ ID NO: 160 - RNA, 21 nt, Artificial
  antisense strand of siRNA
ugugcuucuagccaggcacuu

SEQ ID NO: 161 - RNA, 57 nt, Artificial
  target mRNA
agcagcuaccaaugcugcuugugccuggcuagaagcacaagaggaggaggagguggg

SEQ ID NO: 162 - RNA, 57 nt, Human immunodeficiency virus
agcagcuaccaaugcugcuuguaccuggcuggaagcacaagaggaggaggagguggg

SEQ ID NO: 163 - RNA, 21 nt, Artificial
  2nd generation si/sh RNA
ugugcuuccagccagguacuu

SEQ ID NO: 164 - RNA, 47 nt, Artificial
  shRNA sequence
nnnnnnnnnnnnnnnnnnuucaagagannnnnnnnnnnnnnnnnnuu

SEQ ID NO: 165 - RNA, 19 nt, Human immunodeficiency virus
gugccuggcuugaagcaca

SEQ ID NO: 166 - protein, 8 aa, Human immunodeficiency virus
SYGRKKRR

SEQ ID NO: 167 - DNA, 50 bp, Human immunodeficiency virus
tcctatggcaggaagaagcggagactcctatggcaggaagaagcggagac

SEQ ID NO: 168 - protein, 7 aa, Human immunodeficiency virus
MAGRSGD

SEQ ID NO: 169 - protein, 7 aa, Human immunodeficiency virus
GKQMAGD

SEQ ID NO: 170 - DNA, 52 bp, Human immunodeficiency virus
attatggaaaacagatggcaggtgatattatggaaaacagatggcaggtgat

SEQ ID NO: 171 - protein, 7 aa, Human immunodeficiency virus
MENRWQV